(19) United States
(12) Patent Application Publication
(10) Pub. No.: US 2023/0082631 A1
(43) Pub. Date: Mar. 16, 2023

(54) METHODS AND ASSAYS WITH POPULATIONS OF CELLS

(71) Applicant: StemCell Technologies Canada Inc., Vancouver (CA)

(72) Inventor: Trevor ROGERS, Vancouver (CA)

(21) Appl. No.: 17/800,808

(22) PCT Filed: Feb. 19, 2021
(86) PCT No.: PCT/CA2021/050190

§ 371 Date: Aug. 18, 2022

Related U.S. Application Data
(60) Provisional application No. 62/979,025, filed on Feb. 20, 2020.

(51) Int. Cl. C12N 5/00; G01N 15/14; G01N 33/543

(52) U.S. Cl. CPC C12N 5/0068; G01N 15/1459; G01N 33/54326; C12N 2501/599; C12N 2529/00; G01N 2333/70517; G01N 2333/7055; G01N 2333/70596

(57) ABSTRACT

This disclosure relates to methods for enriching a first population of cells positive for a target moiety and/or a second population of cells positive for the target moiety from a sample, wherein a level of the target moiety among the first population of cells is relatively lower than the level of the target moiety among the second population of cells. The methods of this disclosure may also be adapted to assays for determining distinct populations of cells positive for a target moiety in a sample, and to assays for optimizing enrichment conditions. Last, this disclosure relates to kits of components that may be used to carry out the methods and assays.

This application claims the benefit of U.S. Provisional patent application Ser. No. 62/979,025, filed Feb. 20, 2020, the entire content of which is incorporated herein by reference.

### FIELD

This application relates to the separation of cells, and more particularly to the preferential enrichment of populations of cells within a sample characterized by different levels of a target moiety.

### BACKGROUND

Multicellular organisms comprise vast numbers of cells, and the products of such cells. The cells of a multicellular organism may be classified on various different bases. For example, cells may be distinguished based on their structure and/or function. Or, cells may be categorized depending on their origin, developmental stage, or tissue residency. The categorization of cells may also be facilitated with reliance on gene expression signatures or on the manifestation of gene expression, such as through localization of peptides or proteins to the surface of a cell.

Given the complexity of a multicellular organism and its vast number of cell types, the study of such cells has been greatly aided by cell separation technologies. Early modes of separating cell types were accomplished based on differential adhesion to a substrate, preferential survival/expansion under specified culture conditions, size fractionation, or on differential sedimentation rates such as in a density gradient medium. More recent modes of separating cell types exploit the presence/absence of target moieties, as may be present on the surface of a cell, and the recognition of such target moieties by binding partners.

Immunomagnetic cell separation reagents have been commercialized by STEMCELL Technologies and other companies, which reagents may be used to perform positive or negative cell separations. In positive cell separation, a cell of interest is isolated on the basis that it presents a target moiety of interest. In negative cell separation, a cell of interest is isolated on the basis that a cell of non-interest presents a target moiety of interest. A major distinction between both approaches is that negative cell separation yields “untouched cells”, which may have particular utility in downstream applications. Indeed, sequential separations using positive or negative, or both, approaches are routinely used to yield a cell population of interest.

However, these approaches may fail when a cell population of interest is positive for the same target moiety as cells that are not of interest. Thus, there is a need for improved methods for segregating and/or separating cells based on differential levels of a target moiety.

### SUMMARY

The present disclosure relates to methods for segregating target moiety-positive populations of cells in a sample based on a presented level of a target moiety, and to assays for identifying distinct target moiety-positive populations of cells in a sample based on a presented level of a target moiety.

In one broad aspect of this disclosure are provided methods for enriching (or segregating) a first population of cells positive for a target moiety and/or a second population of cells positive for the target moiety from a sample, comprising labeling the first population and the second population with particles to form cell:particle complexes; contacting the cell:particle complexes with an enrichment reagent to substantially delabel the first population from the particles; and isolating the first population from the sample, wherein the level of the target moiety among the first population of cells is relatively lower than the level of the target moiety among the second population of cells.

In one embodiment, the methods may further comprise contacting residual cell:particle complexes in the sample with a separation reagent to substantially separate the second population from the particles.

In one embodiment, the methods may further comprise isolating the second population from the sample.

In one embodiment, the target moiety is a cell surface marker. In one embodiment, the marker is human CD271, human CD25, human CD49d, mouse CD138, human CD8, or human CD56.

In one embodiment, the particles are coated with a polymer. In one embodiment, the particles are responsive to a magnetic field.

In one embodiment, the methods may further comprise fractionating the cell:particle complexes from the sample after step a) and before step c)

In one embodiment, the methods may further comprise providing at least a saturating quantity of particles relative to the level of the target moiety.

In one embodiment, the labeling of the first population of cells or the second population of cells with the particles is intermediated by antibodies or antibody fragments. In one embodiment, the antibodies or antibody fragments comprise a particle-specific member and a target moiety-specific member. In one embodiment, the particle-specific member is linked, directly or indirectly, to the target moiety-specific member. In one embodiment, the particle-specific member and the target-moiety-specific member form a bispecific complex.

In one embodiment, the enrichment reagent is formulated differently from the separation reagent. In one embodiment, the enrichment reagent and the separation reagent each include a polymer. In one embodiment, a concentration of the polymer is relatively lower in the enrichment reagent compared to the separation reagent. In one embodiment, the polymer is PEG (polyethylene glycol), PEG-based, or PEG-like. In one embodiment, the polymer is dextran, dextran-based, or dextran-like.

In one embodiment, the methods may further comprise enriching the cell:label complexes from the sample after step a) and before step b).

In another broad aspect of this disclosure are provided assays for identifying in a sample distinct populations of cells positive for a target moiety, comprising labeling the target moiety with a particle to form cell:particle complexes; and acquiring by flow cytometry a readout of the cell:particle complexes, wherein the level of the target moiety among the first population of cells is (relatively) lower than the level of the target moiety level among the second population of cells.

In one embodiment, the readout of cell:particle complexes of the first population is distinct from the readout of cell:particle complexes of the second population. In one embodiment, the readout of both the cell:particle complexes of the first population and cell:particle complexes of the second population is distinct from the readout of uncomplexed target moiety-positive cells. In one embodiment, the read-out is a side scatter profile.

In one embodiment, the target moiety is a cell surface marker. In one embodiment, the marker is human CD271, human CD25, human CD49d, mouse CD138, human CD8, or human CD56.

In one embodiment, the particles are responsive to a magnetic field.

In one embodiment, the assays may further comprise providing at least a saturating quantity of particles relative to the level of the target moiety of the first population and the second population.

In one embodiment, the labeling of the first population of cells or the second population of cells with the particles is intermediated by antibodies or antibody fragments. In one embodiment, the antibodies or antibody fragments comprise a particle-specific member and a target moiety-specific member. In one embodiment, the particle-specific member is linked, directly or indirectly, to the target moiety-specific member. In one embodiment, the particle-specific member and the target-moiety-specific member form a bispecific complex.

In one embodiment, the assays may further comprise gating the side scatter profile on the target moiety.

In one embodiment, the assays may further comprise fractionating the cell:particle complexes from the sample before acquiring the readout.

In one embodiment, the assays may further comprise contacting the cell:particle complexes with an enrichment reagent and reacquiring by flow cytometry the readout to assess a shift in the readout. Thus, in another broad aspect of the disclosure are provided assays for measuring dose-responsiveness of cell:particle complexes to the enrichment reagent.

In another broad aspect of this disclosure are provided kits for enriching a first population of cells positive for a target moiety and/or a second population of cells positive for the target moiety from a sample, comprising a tube containing polymer-coated particles; a tube containing an antibody composition, the antibody composition comprising a particle-specific member linked to a target moiety-specific member; a tube containing an enrichment reagent; and optionally, a tube containing a separation reagent.

In one embodiment, the enrichment reagent is PEG-containing or dextran-containing.

In one embodiment, the separation reagent is PEG-containing or dextran-containing.

In one embodiment, a concentration of PEG or dextran is relatively lower in the enrichment reagent than in the separation reagent

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows the results of neural crest cell differentiation of multiple human ES and human iPS cell lines. Various ES and iPS cell lines were maintained in either mTeSR™1 or TeSR™-E8′ and then differentiated using the STEMdiff™ Neural Crest Differentiation Kit (STEMCELL Technologies). A) The human embryonic stem (“ES”) and induced pluripotent stem (“iPS”) cells efficiently differentiated into SOX10⁺ neural crest cells (85.5±1.6%; mean±SEM; n=9). B) Levels of PAX6⁺ neuro-ectodermal cells in the cultures of A) varied in a cell line-dependent manner (5.6±0.7%; mean±SEM; n=9). Numbers are % positive over total DAPI in a tiled image. Dots show results of individual experiments.

FIG. 2 shows flow cytometry plots of CD271 levels among undifferentiated H9 cells maintained in mTeSR™1 (STEMCELL Technologies) and H9 cells differentiated to neural crest cells using the STEMdiff™ Neural Crest Differentiation Kit. On day 6, the cells were disaggregated into a single-cell suspension using ACCUTASE™ (STEMCELL Technologies). Cells were gated on DAPI⁻ viable cells. While the undifferentiated H9 cells and H9 cells differentiated to neural crest cells exhibit relatively consistent expression of the surface antigen CD57, the undifferentiated H9 cells are characterized by lower levels of surface CD271 (A) relative to surface CD271 levels of differentiated neural crest cells (B).

FIG. 3 shows flow cytometry plots of CD49d surface levels among H9 cells or B004 cells differentiated using the STEMdiff™ Neural Crest Differentiation Kit. Cells were gated on DAPI⁻ viable cells. Expression of CD49d^{High} (A) and CD271^{High} (B) among the assayed H9 cells was 90% and 87%, respectively. Expression of CD49d^{High} (C) and CD271^{High} (D) among the assayed B004 cells was 34% and 33%, respectively.

FIG. 4 shows that a single surface antigen, CD271, may discriminate between PAX6⁺ neural cells and SOX10⁺ neural crest cells. F016 cells differentiated in STEMdiff™ Neural Crest Differentiation Kit were analyzed for surface CD271 levels (A) and intracellular SOX10 and PAX6 levels (B). R038 cells differentiated in STEMdiff™ Neural Crest Differentiation Kit were analyzed for surface CD271 levels (C) and intracellular SOX10 and PAX6 levels (D). H1 cells differentiated in STEMdiff™ Neural Crest Differentiation Kit were analyzed for surface CD271 levels (E) and intracellular SOX10 and PAX6 levels (F). Prior to fixation and permeabilization, the cells were labeled using GloCell™ Fixable Viability Dye Violet 450 (STEMCELL Technologies). Viable cells were gated by excluding the GloCell™ Fixable Viability Dye 450 signal. Fluorochrome- and isotype-matched control antibodies were used to determine the baseline fluorescent signals. Each flow cytometry plot was gated to display CD271^{Low} cells (grey dots) or CD271^{High} neural crest cells (black dots). For all three cell lines examined, the SOX10 expression overlapped with the CD271^{High} cells, while PAX6 expression overlapped with the CD271^{Low} cells.

FIG. 5 shows differential expression of CD271 in flow cytometry plots. Neural crest cells were differentiated from 1C and H9 cells using the STEMdiff™ Neural Crest Differentiation Kit. After 6 days in culture the cells were harvested, pooled, and labeled with a PE-conjugated anti-human CD271 antibody. Analysis by flow cytometry of the pooled cell population (A), after fractionation based on expression of the CD271 antigen (B), and after preferential isolation of CD271^{High} cells by treatment of the cells in (B) with an enrichment reagent (C) are shown. The population was gated on viable singlet CD271^{High} cells.

FIG. 6 shows that particles used to label cells increase the side scatter (SSC) signal detected by flow cytometry. (A) H9, 1C and M001 cells were differentiated in STEMdiff™ Neural Crest Differentiation Kit, and arising CD271⁺ cells were fractionated by positive selection (as in FIG. 5B). The enriched CD271⁺ cells were further cultured for 6 days in STEMdiff™ Neural Crest Differentiation Kit then incubated with PE-conjugated antibodies against either the antibody composition or the particles used to fractionate the CD271⁺ cells. Each dot in A) represents either the starting population before positive selection (start) or the different conditions tested for optimizing the cell separation procedure (Experiment 1, 2 and 3), where the same fill-colour represents head-to-head experiments. In B) through D), flow cytometry plots, gated on DAPI⁻ viable cells, show a pooled population of H9 and 1C cells unlabeled with particles ((B) and (D)), or CD271⁺ cells fractionated from the pooled population using antibody compositions and particles ((C) and (E)). CD271 expression was assessed and shown as CD271^{Low} (grey dots) or CD271^{High} (black dots). Unlabeled cells show no difference in SSC⁺ signal between the CD271^{Low} and CD271^{High} cells (D). When the cells are labeled with particles, a relatively larger SSC⁺ signal was associated with the CD271^{High} cells when compared to CD271^{Low}cells (E). (F) CD271^{High} and CD271^{Low} cells from a pooled population of H9 and 1C cells demonstrate differential separation efficiencies depending on the concentration of enrichment reagent (data for n=1 neural crest cell culture).

FIG. 7 shows the effects of different concentrations of enrichment reagents on the purity and recovery of CD271^{High} neural crest cells. 1C or H9 cells were differentiated as in FIG. 5. (A) Fractionated CD271⁺ cells were incubated with different concentrations of PEG-containing enrichment reagent (“first enrichment reagent”), and the purity and recovery of CD271^{High} neural crest cells were compared to cells incubated in a conventional PBS-based wash reagent (“0%”). Shown are results of n=6 different CD271^{High} neural crest cell separations. Mean purity or recovery is represented by “+” symbols in the box-and-whisker plots. Paired two-tailed t-tests were performed on logit-transformed data (p≥0.05 ns, p<0.05*, p<0.01**, p<0.001***). (B) Fractionated CD271⁺ cells were incubated with different concentrations of a dextran-containing enrichment reagent (“second enrichment reagent”), and the purity and recovery of CD271^{High} neural crest cells were compared to cells incubated in a conventional PBS-based wash reagent (“0%”). Shown are results of CD271^{High} neural crest cells separations, where n=5 (0%), n=4 (0.05%), or n=1 (0.01% and 0.5%). Mean purity or recovery is represented by “+” symbols in the box-and-whisker plots. Paired two-tailed t-tests were performed on logit-transformed data (p≥0.05 ns, p<0.01**). (C) An assay was developed to rapidly and accurately determine responsiveness to different concentrations/formulations of enrichment reagent of positively selected target moiety-positive cells that stratify in at least two populations based on the level of the target moiety. The geometric mean of the side scatter signal for cells labeled with particles was determined via flow cytometry analysis. The dose-response curve was generated by plotting log[inhibitor] vs response using a variable slope fit (four parameter). The vertical dotted lines on the dose-response curves represent the calculated IC₅₀ values.

FIG. 8 shows that enrichment reagent may be added at different times during CD271^{High} neural crest cell isolation. (A) The order of addition of antibody composition (“C”), particles (“P”), and enrichment reagent (“E”) was varied during immunomagnetic positive selection of CD271⁺ cells, and % purity and % recovery of CD271^{High} neural crest cells was assessed. Shown are the results from n=4 different neural crest cell cultures. (B) The timing of addition of enrichment reagent—whether before incubating the cells with antibody composition, after the cells are incubated with antibody composition and particles (“After 1^{st} top-up”), or after magnetic separation (“After 1^{st} pour-off”)—was varied during immunomagnetic positive selection of CD271⁺ cells, and % purity and % recovery of CD271^{High} neural crest cells was assessed. Shown are the results from n=19 (enrichment reagent added before the antibody composition), n=6 (enrichment reagent added after the 1^{st} top-up), or n=10 (enrichment reagent added after the 1^{st} pour-off) different neural crest cell cultures. Mean purity or recovery is represented by “+” symbols in the box-and-whisker plots. Paired two-tailed t-tests were performed on logit-transformed data (p≥0.05 ns, p<0.01**, p<0.001***).

FIG. 9 shows differential expression of CD271 in flow cytometry plots. 1C or H9 cells were differentiated using the STEMdiff™ Neural Crest Differentiation Kit. After 6 days in culture the cells were harvested, pooled, and labeled with a PE-conjugated anti-human CD271 antibody. Analysis by flow cytometry of the CD271^{Low} population of cells within the pooled cell population (A) and of the C271^{Low} cells isolated by pour-off after incubation of positively selection CD271 cells with the enrichment reagent (B). The population was gated on viable singlet CD271^{Low} cells. (C) After fractionating the differentiated CD271⁺ cells, the cells were incubated with different concentrations of a PEG-containing enrichment reagent, and the purity and recovery of isolated CD271^{Low} cells were compared to a control, conventional PBS-based wash reagent (“0%”). Shown is the negative pour-off fraction from CD271 positive selection experiments performed with n=3 different neural crest cell cultures. Paired two-tailed t-tests were performed on logit-transformed data (p≥0.05 ns, p<0.05*, p<0.01**). (D) An identical procedure as shown in (C) was performed using dextran-coated particles. During isolation of the differentiated CD271⁺ cells, the cells were incubated with different concentrations of a dextran-containing enrichment reagent, and the purity and recovery of isolated CD271^{Low} cells were compared to control, conventional PBS-based wash reagent (“0%”). Shown is the negative pour-off fraction from CD271 positive selection experiments performed with n=1 neural crest cell culture.

FIG. 10 shows that enriched CD271^{High} neural crest cells are functional. (A) Differentiated and enriched CD271^{High} cells expand in culture, but CD271^{Low} neural cells do not markedly expand in the same culture conditions. H9 cells were differentiated using the STEMdiff™ Neural Crest Differentiation Kit. After 6 days in culture the cells were harvested, pooled, and fractionated by immunomagnetic positive selection of CD271 cells, followed by incubation in enrichment reagent. The enriched CD271^{High} population was plated into STEMdiff™ Neural Crest Differentiation medium and cultured for an additional 7 days. Viable CD271^{High} cells expanded from 3.81×10⁵ to 6.43±0.97×10⁵ cells (mean±SEM; 4 replicate wells). Under the same conditions, only limited expansion of the negative CD271^{Low} fraction was observed, from 1.91×10⁴ to 2.89±1.18×10⁴ viable cells (mean±SEM; 4 replicate wells). (B) Seeding densities of CD271^{High} cells for further culture can be varied. White arrows indicate PAX6⁺ cells, as observed in the non-enriched start culture wells. Isolated populations enriched for CD271^{High} cells formed a confluent layer of SOX10⁺ cells, indicating the establishment of an enriched culture of neural crest cells with minimal PAX6⁺ contaminants. (C) Replated, enriched CD271^{High} neural crest cells may be differentiated to peripheral neurons. Adopting a seeding density of B), peripheral neuron differentiation was induced by passaging neural crest cells at day 6 into the conditions published in Lee G et al. (2010) Nat Protoc 5(4): 688-701. White arrows point to cell bodies expressing Brn3A, a transcription factor found in the nucleus of developing peripheral neurons. The presence of axon connections between peripheral neuron cell bodies was assessed by peripherin expression, a type Ill intermediate filament protein that is expressed in neurons of the peripheral nervous system.

FIG. 11 shows that CD25^{High} cells may be preferentially enriched from leukapheresis samples. Analysis by flow cytometry of the leukapheresis sample for CD25 cells (A), of CD25⁺ cells fractionated by immunomagnetic positive selection (B), and enrichment of CD25^{High} cells from the population of (B) using enrichment reagent (C). In each of (A)-(C) the population was gated on viable singlet CD45⁺CD25^{High} cells. (D) Delabeling of enriched CD25⁺ cells by different concentrations of enrichment reagent. The geometric mean of the side scatter signal for cells labeled with polymer-coated particles was determined by flow cytometry analysis. The dose-response curve was generated by plotting log[inhibitor] vs response using a variable slope fit (four parameter). (E) CD25⁺ cells fractionated by immunomagnetic positive selection were incubated with different concentrations of a PEG-containing enrichment reagent or a control, and the purity and recovery of CD25^{High} cells were compared to a control, conventional PBS-based wash reagent (“0%”). Shown are results from n=3 leukapheresis donors, where the same fill-colour represents head-to-head experiments. Paired two-tailed t-tests were performed on logit-transformed data (p≥0.05 ns, p<0.05*, p<0.01**). (F) The enriched CD25^{High} cells of (E) were further incubated with an anti-CD127 depletion reagent. The purity and recovery of delabeled FOXP3⁺CD25^{High} regulatory T cells were compared to a control, conventional PBS-based wash reagent (“0%”). Shown are results from n=4 leukapheresis donors. Mean purity or recovery is represented by “+” symbols in the box-and-whisker plots. Paired two-tailed t-tests were performed on logit-transformed data (p≥0.05 ns, p<0.05*, p<0.01**).

FIG. 12 shows that CD56^{High} cells may be preferentially enriched from leukapheresis samples. Analysis by flow cytometry of the leukapheresis samples for CD56 cells (A), of CD56⁺ cells fractionated by immunomagnetic positive selection (B), and enrichment of CD56^{High} cells from the population of (B) using enrichment reagent (C). In each of (A)-(C) the population was gated on viable singlet CD45⁺CD56^{High} cells. (D) Delabeling of enriched CD56⁺ cells by different concentrations of enrichment reagent. The geometric mean of the side scatter signal for cells labeled with polymer-coated particles was determined by flow cytometry analysis. The dose-response curve was generated by plotting log[inhibitor] vs response using a variable slope fit (four parameter). (E) CD56⁺ cells fractionated by immunomagnetic positive selection were incubated with different concentrations of a PEG-containing enrichment reagent or a control, and the purity and recovery of CD56^{High} cells were compared to a control, conventional PBS-based wash reagent (“0%”). Shown are the results from n=4 leukapheresis donors. Mean purity or recovery is represented by “+” symbols in the box-and-whisker plots. Paired two-tailed t-tests were performed on logit-transformed data (p≥0.05 ns, p<0.05*, p<0.01**).

FIG. 13 shows that CD8^{High} cells may be preferentially enriched from leukapheresis samples. Analysis by flow cytometry of the leukapheresis samples for CD8 cells (A), of CD8⁺ cells fractionated by immunomagnetic positive selection (B), and enrichment of CD8^{High} cells from the population of (B) using enrichment reagent (C). In each of (A)-(C) the population was gated on viable singlet CD45⁺CD8^{High} cells. (D) Delabeling of enriched CD8⁺ cells by different concentrations of enrichment reagent. The geometric mean of the side scatter signal for cells labeled with polymer-coated particles was determined by flow cytometry analysis. The dose-response curve was generated by plotting log[inhibitor] vs response using a variable slope fit (four parameter). (E) CD8⁺ cells fractionated by immunomagnetic positive selection were incubated with different concentrations of a PEG-containing enrichment reagent or a control, and the purity and recovery of CD8^{High} cells were compared to a control, conventional PBS-based wash reagent (“0%”). Shown are the results from n=1 leukapheresis donor.

FIG. 14 shows that CD138^{High} cells may be preferentially enriched from naïve mouse splenocyte samples. Analysis by flow cytometry of the mouse splenocyte samples for CD138 cells (A), of CD138⁺ cells fractionated by immunomagnetic positive selection (B), and enrichment of CD138^{High} cells from the population of (B) using enrichment reagent. In each of (A)-(C) the population was gated on viable singlet CD45⁺CD267 (TACI)⁺CD138^{High} plasma cells/blasts. (D) Delabeling of enriched CD138⁺ cells by different concentrations of enrichment reagent. The geometric mean of the side scatter signal for cells labeled with polymer-coated particles was determined by flow cytometry analysis. The dose-response curve was generated by plotting log[inhibitor] vs response using a variable slope fit (four parameter). (E) CD138⁺ cells fractionated by immunomagnetic positive selection were incubated with different concentrations of a PEG-containing enrichment reagent or a control, and the purity and recovery of CD138^{High} cells were compared to a control, conventional PBS-based wash reagent (“0%”). Shown are results from n=6 different mouse splenocyte samples. Mean purity or recovery is represented by “+” symbols in the box-and-whisker plots. Paired two-tailed t-tests were performed on logit-transformed data (p≥0.05 ns, p<0.05*, p<0.01**, p<0.001***). (F) CD138⁺ cells fractionated by immunomagnetic positive selection were incubated with different concentrations of a dextran-containing enrichment reagent or a control, and the purity and recovery of CD138^{High} cells were compared to a control, conventional PBS-based wash reagent (“0%”). Shown are results from n=2 (0% and 0.04%) and n=1 (0.08% and 0.16%) different mouse splenocyte samples.

FIG. 15 shows that CD138^{High} cells may be preferentially enriched from naïve C57BL/6 mouse bone marrow samples. Analysis by flow cytometry of the mouse bone marrow samples (A), of CD138⁺ cells fractionated by immunomagnetic positive selection (B), and enrichment of CD138^{High} cells from the population of (B) using enrichment reagent (C). In each of (A)-(C) the population was gated on viable singlet CD45⁺CD267 (TACI)⁺CD138^{High} plasma cells/blasts. (D) CD138⁺ cells fractionated by immunomagnetic positive selection were incubated with different concentrations of a PEG-containing enrichment reagent or a control, and the purity and recovery of CD138^{High} cells were compared to a control, conventional PBS-based wash reagent (“0%”). Shown are results from n=6 different mouse bone marrow cell samples. Mean purity or recovery is represented by “+” symbols in the box-and-whisker plots. Paired two-tailed t-tests were performed on logit-transformed data (p≥0.05 ns, p<0.05*, p<0.01**, p<0.001***). (E) CD138⁺ cells fractionated by immunomagnetic positive selection were incubated with different concentrations of a dextran-containing enrichment reagent or a control, and the purity and recovery of CD138^{High} cells were compared to a control, conventional PBS-based wash reagent (“0%”). Shown are results from n=2 (0% and 0.04%) and n=1 (0.08% and 0.16%) different mouse bone marrow cell samples.

### DETAILED DESCRIPTION

This disclosure relates to methods, assays and kits in regards to distinct target moiety-positive populations of cells in a sample. More specifically, the distinct populations of cells positive for a target moiety comprise a first population of cells positive for a target moiety and a second population of cells positive for the target moiety. In one embodiment, a target moiety level of the first population of cells positive for the target moiety is (relatively) lower than the target moiety level of the second population of cells positive for the target moiety.

Where used herein, the term “target moiety” refers to a cell-associated motif, the presence, absence or amount (i.e. level) of which may be exploited to help identify or classify a specific type of cell or population of cells. In one embodiment, the target moiety is a cell surface marker. In some cases, a target moiety may uniquely identify a specific cell population (i.e. cell type). For example, CD45 uniquely associates with normal human hematopoietic cells. In some cases, a target moiety does not uniquely identify a specific cell population (i.e. cell type). For example, CD8 associates with at least normal human NK cells and normal human T cells. In such cases, to distinguish among cell types, it may be necessary to further ascertain the presence or absence of a different target moiety. For example, CD8 associates with at least normal human NK cells and normal T cells, but further querying CD56 may distinguish between such populations. In some cases, a level of the target moiety may also be used to distinguish between two or more target moiety-positive populations of cells. For example, human CD271 expression associates with both cells of the neural crest cell lineage (CD271^{High}) and cells of the neuroectodermal and non-neural ectodermal cell lineages (CD271^{Low}). Likewise, different human cell populations may also be stratified based on levels of CD25, CD49d, CD8, or CD56. In the mouse, the level of CD138 may be queried to stratify different target moiety-positive populations of cells in a sample.

Where used herein, the term “sample” refers to a preparation of cells, such as a suspension of cells. The preparation of cells may be of any species, any developmental stage, any tissue type, or any disease state. The sample of cells will include a first population of cells positive for a target moiety, a second population of cells also positive for the target moiety, and may include a population of cells negative for the target moiety. In some embodiments, the cells are vertebrate cells. In some embodiments, the cells are mammalian cells. In some embodiments, the cells are human cells. The preparation of cells may be obtained by processing an organ or a tissue, such as by means conventional in the life sciences arts. Or, the preparation of cells may be obtained from a solution, such as blood, which solution may be pre-processed to remove certain contaminants, such as red blood cells or plasma components. The preparation of cells may also be obtained from an in vitro or ex vivo culture of cells. The in vitro or ex vivo culture of cells may be a maintenance or expansion culture, or the in vitro or ex vivo culture of cells may be a differentiated culture of cells. In some cases, the preparation of cells may be a non-adherent (e.g. suspension) culture of cells, in which case such preparation of cells may be ready for use in the methods or assays disclosed herein, or such cells may be subjected to one or more pre-processing steps, such as to remove contaminants, before they are ready for use. In some cases, the preparation of cells may be adherent cells, in which case such cells may need to be liberated from a cell culture substrate, using conventional techniques, before they are ready for use in the methods or assays disclosed herein.

Where used herein, the term “particle” refers to a body that may be used to mark, label or bind a cell, such as a target moiety-positive cell. In some embodiments, the characteristics of the particle may be exploited in downstream detection or isolation assays. Thus, the characteristics of the particle may dictate one or more fractionation means (such as in response to a magnetic field or on the basis of density (e.g. specific gravity/relative density). Non-limiting examples of downstream assays may include, separating or imaging the cell(s) having been marked/labeled by the particle. In some embodiments, the particle may be magnetic, paramagnetic, or superparamagnetic, and may thus be adopted into workflows utilizing a magnetic field to facilitate separation of the cell(s) labeled with the particle(s). In some cases, the particle may be functionalized in order to facilitate downstream assays, such as isolation or detection assays. For example, particles are routinely conjugated with anti-target moiety antibodies or antibody fragments. Or, particles may be functionalized with different types of coatings, which coatings may be capable of being bound by antibodies or antibody fragments, or antibody compositions that link the particle to target moiety positive cells. For example, particles may be functionalized with avidin or streptavidin, and such particles may be complexed to biotin or biotinylated-entities (e.g. biotinylated anti-target moiety antibodies or antibody fragments). Alternatively, other coatings may functionalize particles, and in one specific embodiment, the coating may be a polymer. Examples of polymer coatings include PEG, PEG-based, or PEG-like coatings. Or, examples of polymer coatings include dextran, dextran-based, or dextran-like coatings.

### Methods

In one aspect of this disclosure, the methods encompass those steps for enriching target moiety-positive populations of cells in a sample based on a target moiety level of such cells. More specifically, the target moiety-positive cells within the sample include at least a first population of cells positive for a target moiety and a second population of cells positive for the target moiety, wherein a target moiety level of the first population of cells is relatively (or on average) lower than the target moiety level of the second population of cells.

The methods may begin by providing a sample of cells. The sample of cells may originate from any source, but in preferred embodiments, the sample is a single-cell suspension of cells. In some embodiments, the sample of cells may include or consist of relatively small clusters or clumps of cells, or the sample of cells may include or consist of relatively large aggregates of cells.

In many cases, prior to providing the sample of cells, they may be pre-processed to break down tissues or organs into constituent parts, to clarify solutions comprising cells of certain contaminants therein, or to detach the cells from a substrate.

In embodiments where tissues or organs are the starting point, methods of dissociating the tissue or organ into single cells are known. For example, the tissue or organ may be digested enzymatically, disrupted mechanically, or disaggregated chemically. In some cases, processing a tissue or organ may involve any combination of enzymatic digestion, mechanical disruption, and chemical disaggregation to yield a suitable suspension of cells.

In embodiments where solutions comprising cells are the starting point, such solutions may be clarified to remove contaminants and the like. Methods of clarifying such solutions are known, and include filtration or chemical treatment. For example, if the starting cells are to be obtained from a blood sample it may need to be cleared of red blood cells and/or other types of cells of non-interest. In one embodiment, a blood sample may be clarified by density gradient centrifugation. In one embodiment, agglutinating red blood cells may clarify a blood sample, such as by performing a rosetting (RosetteSep™ STEMCELL Technologies) protocol. In one embodiment, a blood sample may be clarified by lysing red blood cells with a lysis buffer. In one embodiment, a blood sample may be clarified by changing the osmotic pressure within the red blood cells. If the solution comprising cells is a bodily fluid other than blood, such as urine, it may be important to concentrate the cells therein, while also clarifying the solution of non-cellular components.

In some embodiments, the starting cells may have previously been in culture. If grown in suspension (i.e. under non-adherent conditions), the cells may be ready for use in the methods disclosed herein. However, cells grown in suspension are typically bathed in a cell culture medium and it may be appropriate to pellet the cells and resuspend them in an appropriate physiological buffer, such as phosphate buffered saline or EasySep™ Buffer (STEMCELL Technologies). Further, a suspension of cells bathed in a cell culture medium may not be at an optimal density, which may be readily adjusted by centrifugation and resuspension, such as in an appropriate buffer.

In some embodiments, the starting cells may have been previously cultured adhered to a substrate, whether a tissue culture dish or flask, or to microcarriers. In such embodiments, it is necessary to detach the cells from the substrate, and those skilled in the art will know this may be done by mechanical, enzymatic, or chemical means. Whether the cells are mechanically, enzymatically, or chemically detached from a substrate, or detached using a combination of any of these methods, adjusting the density of the detached cells may also be necessary prior to initiating the methods disclosed herein. When detaching the cells, care should be taken to minimize or avoid excessive exposure to said agent to limit digestion of a target moiety (and/or other moieties) on the cell surface.

The methods disclosed herein encompass a broad range of cell densities. In one embodiment, the cell density in the sample should be between about 1×10⁴ cells/mL and 1×10¹⁰ cells/mL, or between about 1×10⁹ cells/mL and 1×10⁹ cells/m L, or between about 5×10⁹ cells/mL and 5×10⁸ cells/mL. In one embodiment, the cell density in the sample should be about 5×10⁷±0.5×10⁷ cells/mL.

Once the sample of cells is adjusted to an appropriate density and/or suspended in an appropriate buffer, target moiety-positive cells within the sample are labeled with particles to form cell:particle complexes.

In one embodiment, the target moiety is a cell surface marker. Many cell surface markers are known, and are thus encompassed by this disclosure. The target moiety should be bindable by a binding member, such as an antibody or antibody fragment. In one embodiment, populations of cells of interest within a sample are characterized by unique or distinguishable target moiety levels. For example, a target moiety level of a first population of cells (positive for a target moiety) is relatively lower than the target moiety level of a second population of cells (also positive for the target moiety).

In one embodiment, the target moiety is human CD271, wherein CD271^{High} distinguishes neural crest cells and CD271^{Low} distinguishes neuroectodermal and non-neural ectodermal cells.

In one embodiment, the target moiety is human CD49d, wherein CD49d^{High} distinguishes neural crest cells and CD49d^{Low} distinguishes neuroectodermal and non-neural ectodermal cells.

In one embodiment, the target moiety is human CD25, wherein CD25^{High} distinguishes regulatory T and activated T cells and CD25^{Low} distinguishes non-activated T cells.

In one embodiment, the target moiety is human CD8, wherein CD8^{High} distinguishes T cells and CD8^{Low} distinguishes a subset of natural killer cells.

In one embodiment, the target moiety is human CD56, wherein CD56^{High} distinguishes cytokine-producing subsets of natural killer cells and CD56^{Low} distinguishes cytotoxic subsets of natural killer cells.

In one embodiment, the target moiety is mouse CD138, wherein CD138^{High} distinguishes terminally differentiated plasmablasts and plasma cells and CD138^{Low} distinguishes pre-B cells.

It will be clear to the skilled reader that the methods disclosed herein are not limited to the target moieties specified above, but are generalizable to any system where different populations of cells in a sample may be distinguished based on respective levels of a target moiety.

Labeling the first population of cells positive for a target moiety and the second population of cells positive for the target moiety with particles (to form cell:particle complexes) may be accomplished in numerous ways. In one embodiment, a connection between the particles and the first population of cells and the second population of cells, respectively, is intermediated by antibodies or antibody fragments. In one embodiment, the antibodies or antibody fragments comprise a particle-specific member and a target moiety-specific member.

Antibodies or antibody fragments of this disclosure may correspond to any structure capable of binding a target moiety. In one embodiment, the antibodies may correspond to any isotype, including IgA, IgD, IgE, IgG, and IgM. In one embodiment, the antibody fragments are F(ab), F(ab′)2, scFv fragments, or any adaptations thereof. In some embodiments, the antibodies or antibody fragments bind a target moiety with a high degree of specificity. Thus, it is preferred that the antibodies or antibody fragments are monoclonal.

In one embodiment, labeling of the target moiety with a particle is mediated by a single antibody, or fragment thereof. In such an embodiment, one arm of the single antibody or fragment thereof, binds the particle (i.e. the particle-specific member) and the other arm binds the target moiety (i.e. the target moiety-specific member).

In one embodiment, labeling of the target moiety with a particle is mediated by more than one antibody, or fragment thereof. In one embodiment, a particle-specific member is linked, directly or indirectly, to the target moiety-specific member. In one embodiment, a particle-specific member and the target-moiety-specific member form a bispecific complex.

In one embodiment, the bispecific complex includes a particle-specific member bound directly to a target moiety-specific member. In such an embodiment, a region of the particle-specific member may be bound directly to a region of the target moiety-specific member. For example, the Fc region of the particle-specific member may be directly conjugated to the Fc region of the target moiety-specific member. In the case of antibody fragments, a linking portion of the particle-specific member may be directly conjugated to a linking portion of the target moiety-specific member

In one embodiment, the bispecific complex includes a particle-specific member linked indirectly to a target moiety-specific member. In such an embodiment, indirect linkage of the particle-specific member and the target moiety-specific member can be accomplished in any way. For example, the particle-specific member and the target moiety-specific member may each be bound to a common element. For example, the common element may be a polymer capable of being conjugated with antibodies or antibody fragments. Or, the common element may be a particle or a bead. Or, the common element may be a complementary pair of entities, such as biotin and avidin/streptavidin, where each entity is conjugated to one of the particle-specific member or the target moiety-specific member. In one embodiment, the particle-specific member and the target moiety-specific member may be linked in an immunological complex, wherein they are linked by one or more antibodies or antibody fragments. In one embodiment, the bispecific complex comprises two antibodies or F(ab′)2 fragments thereof—a particle-specific member and a target-moiety-specific member—that are linked in any way as described herein.

The methods may further comprise providing at least a saturating quantity of particles relative to the target moiety level (i.e. the sum of target moieties among the first population of cells positive for a target moiety and the second population of cells positive for the target moiety). Thus, a sufficient quantity of particles may enhance recovery of target moiety-positive cells in a sample.

In one embodiment, the particles are coated with a polymer. In such an embodiment, the polymer may be PEG, PEG-based, or PEG-like. Or, the polymer may be dextran, dextran-based, or dextran-like. Regardless of the nature of the polymer coating the particles, the particle-specific member should be specific for the polymer (of the particle) rather than the particle per se.

The particles may possess qualities that facilitate fractionating cell:particle complexes from other cells in a sample that are not positive for the target moiety or are positive for the target moiety but have been delabeled (as described below). In one embodiment, the particles are responsive to a magnetic field. In such an embodiment, cell:particle complexes (connected by antibodies or antibody fragments) formed in a sample may be exposed to a magnetic field, as typically done in immunomagnetic separations, to fractionate the cell:particle complexes from other cells in the sample that are not positive for the target moiety (or are positive for the target moiety but have been delabeled). In one embodiment, the particles are responsive to a density of a solution in which the cells are suspended. For example, the particles may have a density that is lower than a density of the solution, in which case cell:particle complexes will float in the solution. Or, the particles may have a density that is higher than a density of the solution and cells not included in cell:particle complexes, in which case cell:particle complexes will sink in the solution. Thusly, cell:particle complexes can be fractionated from other cells in the sample that are not positive for the target moiety (or are positive for the target moiety but have been delabeled). Particles as contemplated in this disclosure are available from various suppliers and/or manufacturers, including STEMCELL Technologies. Accordingly, an early or preliminary step of the methods disclosed herein may comprise fractionating the cell:particle complexes from the sample after connecting particles and cells positive for a target moiety (via antibodies or antibody fragments).

To enrich (and eventually isolate) a population of target moiety positive cells characterized by a distinct target moiety level, formed cell:particle complexes (whether or not a fractionation step is performed) are contacted with an enrichment reagent. The cell:particle complexes should be incubated with an enrichment reagent for a sufficient duration of time to substantially delabel the first population of cells positive for a target moiety. As used herein, “substantially delabel” means to cause (via the enrichment reagent) the delabeling or unbinding of particles from a majority, if not all, of the target moieties of the first population of cells. Even though the first population of cells will be characterized by a target moiety level that is relatively lower than the second population of cells, within the first population there may be a spectrum of target moiety level among cells. Thus, if not all, a significant proportion of the first population of cells will be delabeled of particles, or they may retain only a sub-threshold level of particles to render them unresponsive to the fractionation means (as described above). In one embodiment, after incubating the cell:particle complexes in enrichment reagent, about 100%, or about 95%, or about 90%, or about 80%, or about 70%, or about 60%, or about 50% of target moieties of the first population of cells may be delabeled of particles. Overall, the effect of the enrichment reagent is to substantially delabel the first population of cells in preference to the second population of cells, thereby rendering the first population of cells differentially susceptible (i.e. unresponsive) to the fractionation means in comparison to the second population of cells (retained within cell:particle complexes). As such, the first population of cells may be segregated or enriched from the second population of cells, which has previously not been possible when performing immunomagnetic separation of cells.

In one embodiment, the enrichment reagent segregates/enriches the first population from the second population, whereby following incubation of the cell:particle complexes in the presence of enrichment reagent the second population may be retained in proximity of a magnetic field, while the first population is not responsive to the magnetic field.

An enrichment reagent should be gentle on cells and non-toxic to cells (e.g. it should not directly lead to cell death). For example, an enrichment reagent should possess a physiological (with respect to ex vivo animal cells, and more specifically mammalian cells) pH, osmolarity, osmolality, temperature, etc. Further, an enrichment reagent may contain salts and other minerals that are commonly encountered by cells, but only at physiological or approximately physiological levels.

In one embodiment, an enrichment reagent may include a buffer appropriate for bathing mammalian cells, such as phosphate buffered saline, HEPES, MOPS, or Hank's Balanced Salt Solution. In one embodiment, an enrichment reagent may further comprise fetal bovine serum (FBS) and/or bovine serum albumin (BSA) and or serum-derived or recombinant albumin from any species. Where FBS is included in an enrichment reagent, the concentration of FBS in the buffer-based enrichment reagent may be about 40%, about 35%, about 30%, about 25%, about 20%, about 15%, about 10%, about 5%, about 1%, about 0.5% or less. Where BSA or albumin from another species is included in an enrichment reagent, the concentration of albumin in the buffer-based enrichment reagent may be about 10%, about 9%, about 8%, about 7%, about 6%, about 5%, about 4%, about 3%, about 2%, about 1%, about 0.5%, or about 0.1% or less.

In one embodiment, an enrichment reagent may include a chelator appropriate for a solution that is used to bathe cells, such as EDTA. Where a chelator is included in an enrichment reagent, the concentration of the chelator in the buffer-based enrichment reagent may be about 10 μM, between about 5 μM and 10 μM, between about 1 μM and 5 μM, between about 0.5 μM and 1 μM, between about 0.1 μM and 0.5 μM, between about 50 mM and 100 mM, between about 10 mM and 50 mM, between about 5 mM and 10 mM, between about 1 mM and 5 mM or less.

In one embodiment, an enrichment reagent includes one or more polymers, and such one or more polymers should be gentle on cells and non-toxic to cells at the concentrations used. In one embodiment, the polymer is PEG, PEG-based, or PEG-like. In one embodiment, the polymer is dextran, dextran-based, or dextran-like. A polymer included in an enrichment reagent should be the same as or structurally equivalent, at least at the monomer level, to the polymer used to coat the particles.

In one embodiment, the concentration of the one or more polymers in the enrichment reagent should be non-toxic to cells. Accordingly, the polymer should be included in the enrichment reagent at a relatively low concentration (in comparison to a separation reagent, as described below). In one embodiment, the polymer may be included in the enrichment reagent at the minimum concentration necessary to effect the segregation of a first target moiety-positive population of cells from retained cell:particle complexes comprising a second target moiety-positive population of cells. In one embodiment, the concentration of the polymer in the enrichment reagent achieves an IC₅₀, as may be determined via an assay as disclosed herein below. In one embodiment, the concentration of the polymer in the enrichment reagent is less than 10% (w/v), less than 5% (w/v), less than 1% (w/v), less than 0.5% (w/v), less than 0.25% (w/v), less than 0.125% (w/v); less than 0.0625% (w/v); less than 0.03125% (w/v), less than 0.015625% (w/v); or less than 0.01% (w/v).

In one embodiment, components of the enrichment reagent may be pre-formulated into a stock solution, which may be diluted by an end user as appropriate to effect the segregation of a first population of cells positive for a target moiety from a second population of cells positive for a target moiety. In one embodiment, components of the enrichment reagent may be separately formulated, such as into a base solution (e.g. a buffer, which may or may not include FBS and/or BSA and/or serum-derived or recombinant albumin from any species and/or a chelator) and a concentrated active ingredient solution. In such an embodiment, the active ingredient solution is diluted in the base solution to an appropriate concentration, as may be determined using an assay as disclosed herein below or by simple titration experiments.

Depending on the cell type (and the associated target moiety), it may be necessary to optimize dose-responsiveness of enrichment regent concentrations to enhance segregation/enrichment efficiency, and therefore, enrichment of a first population of cells positive for a target moiety from a second population of cells positive for the target moiety. For example, higher concentrations of enrichment reagent may be required when the first population of cells and the second population of cells in a sample both present high, although different, levels of the target moiety. On the other hand, lower concentrations of enrichment reagent may be sufficient when at least the first population of cells, and possibly the second population of cells in a sample, present low (and different) levels of the target moiety.

After contacting the cell:particle complexes with enrichment reagent, the first population of cells can be isolated from the sample. Isolation of the first population of cells is possible because upon being substantially delabeled of particles, a significant proportion of such cells are no longer responsive to the fractionation means (as applicable based on the quality of the particles). However, the second population of cells continue to reside within cell:particle complexes and thus remain susceptible to the influence of the particles. For the sole purpose of clarifying the foregoing, but not intended as limiting, in embodiments where the particles are responsive to a magnetic field, the substantially delabeled first population of cells can be isolated in a negative fraction, while the second population of cells (within cell:particle complexes) are retained while in the presence of a magnetic field.

Prior to isolating the substantially delabeled first population of cells from the sample, in some applications it may be important to fractionate the cell:particle complexes from the sample. An initial fractionation step may occur after the cell:particle complexes are formed, and preferably occurs before the cell:particle complexes are contacted with the enrichment reagent. The fractionation may be performed as described above, such as by taking advantage of the qualities of the particle (whether buoyant, dense, or responsive to a magnetic field). An initial fractionation may be particularly important where a substantially pure first population of cells is the desired output of the disclosed methods, because otherwise the substantially delabeled first population of cells isolated from the sample will be included among cells that are not positive for the target moiety. However, if the second population of cells is the desired output of the disclosed methods, then it may not be necessary to perform such an initial fractionation.

In a specific embodiment, the cell:particle complexes may be formed in a tube using particles responsive to a magnetic field and antibody complexes, comprising a target moiety-specific member and a particle-specific member, the antibody complexes not directly conjugated to the particles. An optional fractionation step may involve positioning the tube in proximity of a magnetic field, and cells in the sample not positive for the target moiety may be poured away while retaining cell:particle complexes. The cell:particle complexes may be resuspended in a buffer, such as the enrichment reagent, with the tube either in proximity or not in proximity of the magnetic field. Alternatively, unfractionated cell:particle complexes may similarly be contacted with the enrichment reagent. Nevertheless, the first population of cells will be substantially delabeled of particles and may similarly be poured away from retained (or residual) cell:particle complexes comprising the second population of cells. Alternatively, the foregoing methods can be performed using columns rather than tubes. Variations on these approaches will be apparent to the person skilled in the art depending on the type of vessel used, whether it is a tube, dish, flask, column, bag, or other type of container.

The enrichment reagent may be added at any time before the first population of cells positive for a target moiety is isolated. More specifically, the enrichment reagent may be added before or after the cell:particle complexes have formed. Still more specifically, the enrichment reagent may be added before or after the particles or bispecific complexes are added to the sample.

Upon isolating the substantially delabeled first population of cells positive for a target moiety, such population is segregated/enriched from the second population of cells positive for the target moiety. Similarly, the second population of cells positive for the target moiety has been fractionated from the first population. On the one hand, if only the first population of cells is desired for downstream applications, then the cell:particle complexes (and free particles after substantially delabeling the first population) retained in the vessel may be discarded. On the other hand, if the second population of cells within residual cell:particle complexes is desired for downstream applications they may also be isolated. Naturally, in some cases it may be desirable to use both the first population of cells and the second population of cells separately in downstream applications.

After the substantially delabeled first population of cells positive for a target moiety are isolated from the sample, the residual cell:particle complexes (comprising the second population of cells) may be subjected to further treatment. In one embodiment, the residual cell:particle complexes may be isolated by removing them from the effects of the fractionation means (e.g. a magnetic field, or otherwise). In one embodiment, it may be desirable to substantially separate the second population of cells by contacting the residual cell:particle complexes with a separation reagent. Where used herein, the phrase “substantially separate” carries, in essence, the same meaning as provided for the phrase “substantially delabel”, except with necessary modification to specifically apply to the separation reagent and the second population of cells positive for a target moiety.

A separation reagent should be gentle on cells and non-toxic to cells (e.g. it should not directly lead to cell death). For example, a separation reagent should possess a physiological (with respect to ex vivo animal cells, and more specifically mammalian cells) pH, osmolarity, osmolality, temperature, density, etc. Further, a separation reagent may contain salts and other minerals that are commonly encountered by cells.

In one embodiment, a separation reagent may include a buffer appropriate for bathing mammalian cells, such as phosphate buffered saline, HEPES, MOPS, or Hank's Balanced Salt Solution. In one embodiment, a separation reagent may further comprise fetal bovine serum (FBS) and/or bovine serum albumin (BSA) and/or serum-derived or recombinant albumin from any species. Where FBS is included in a separation reagent, the concentration of FBS in the buffer-based enrichment reagent may be about 40%, about 35%, about 30%, about 25%, about 20%, about 15%, about 10%, about 5%, about 1%, about 0.5% or less. Where BSA or albumin from any species is included in a separation reagent, the concentration of albumin in the buffer-based enrichment reagent may be about 10%, about 9%, about 8%, about 7%, about 6%, about 5%, about 4%, about 3%, about 2%, about 1%, about 0.5%, or about 0.1% or less.

In one embodiment, a separation reagent may include a chelator appropriate for a solution that is used to bathe cells, such as EDTA. Where a chelator is included in a separation reagent, the concentration of the chelator in the buffer-based separation reagent may be about 10 μM, between about 5 μM and 10 μM, between about 1 μM and 5 μM, between about 0.5 μM and 1 μM, between about 0.1 μM and 0.5 μM, between about 50 mM and 100 mM, between about 10 mM and 50 mM, between about 5 mM and 10 mM, between about 1 mM and 5 mM or less.

In one embodiment, a separation reagent may include one or more polymers, and such one or more polymers should be gentle on cells and non-toxic to cells at the concentrations used. In one embodiment, the polymer is PEG, PEG-based, or PEG-like. In one embodiment, the polymer is dextran, dextran-based, or dextran-like. A polymer included in a separation reagent should be the same as or structurally equivalent, at least at the monomer level, to the polymer used to coat the particles.

In one embodiment, the concentration of the one or more polymers in a separation reagent should be non-toxic to cells. Accordingly, the polymer should be included in a separation reagent at a relatively low concentration. In one embodiment, the polymer may be included in a separation reagent at the minimum concentration necessary to effect the separation of the particles and the second population of cells positive for a target moiety. In one embodiment, the concentration of the polymer in the separation reagent achieves an IC₅₀, as may be determined via an assay as disclosed herein below. In one embodiment, the concentration of the polymer in the separation is less than 10% (w/v), less than 5% (w/v), less than 1% (w/v), less than 0.5% (w/v), less than 0.25% (w/v), less than 0.125% (w/v); less than 0.0625% (w/v); less than 0.03125% (w/v), less than 0.015625% (w/v); or less than 0.01% (w/v).

In one embodiment, components of the separation reagent may be pre-formulated into a stock solution, which may diluted by an end user as appropriate to effect the separation of the second population of cells positive for a target moiety from the particles. In one embodiment, components of the separation reagent may be separately formulated, such as into a base solution (e.g. a buffer, which may or may not include FBS and/or BSA and/or serum-derived or recombinant albumin from any species and/or a chelator) and a concentrated active ingredient solution. In such an embodiment, an end user may add an appropriate volume of active ingredient solution into an appropriate volume of base solution to obtain an appropriately formulated separation reagent, as may be determined using an assay as disclosed herein below or by simple titration experiments.

Depending on the cell type (and the associated target moiety), it may be necessary to measure dose-responsiveness of different concentrations of separation reagent in order to optimize separation of label from a second population of cells positive for a target moiety. For example, higher concentrations of separation reagent may be required when the second population of cells present high levels of target moiety. Since separation reagent is gentle on cells and non-toxic to cells, there may be few limits on the concentration of its components therein. However, as overly high concentrations of separation reagent may be unnecessary and/or lead to unintended consequences, whether for the cells or in downstream applications, a relatively lower concentration of separation reagent may be sufficient for the purpose of separating most, if not all, particles from the second population of cells (within residual cell:particle complexes). Specifically, lower concentrations of separation reagent may be sufficient when the second population of cells present low levels of target moiety.

Thus, enrichment reagent preferentially effects a substantial delabeling of particles from a first population of cells positive for a target moiety, and separation reagent effects a substantial separation of particles from a second population of cells positive for the target moiety (within residual cell:particle complexes). Despite the functional difference of enrichment reagent relative to separation reagent, each reagent may comprise many, or substantially all, of the same components. For example, each reagent may be formulated from a common base solution. In one embodiment, the base solution may be water. In one embodiment, the base solution may be a buffer, such as phosphate buffered saline, HEPES, MOPS, or Hank's Balanced Salt Solution. In one embodiment, each reagent may further comprise a chelator, such as EDTA.

Nevertheless, given that the enrichment reagent effects a preferential enrichment of a first population of cells positive for a target moiety in a sample that also comprises a second population of cells positive for the target moiety, the respective formulations of enrichment reagent and separation reagent may be different in one or more respects. In one embodiment, enrichment reagent is formulated differently from the separation reagent in at least one respect. For example, enrichment reagent and separation reagent may comprise different concentrations of the same active ingredient. In one embodiment, the active ingredient is a polymer; thus in one embodiment a concentration of the polymer is relatively lower in the enrichment reagent compared to the separation reagent. In one embodiment, the polymer is PEG, PEG-based, or PEG-like. In one embodiment, the polymer is dextran, dextran-based, or dextran-like.

Based on the foregoing, the methods of this disclosure encompass those steps for segregating/enriching distinct target moiety-positive populations of cells in a sample based on a target moiety level thereof; the level of the target moiety of a first population of cells being relatively (or on average) lower than the level of the target moiety of a second population of cells.

Adapting the segregation methods of this disclosure, it may be desired to segregate, separate or enrich different populations of cells characterized by different levels of both first and second target moieties. For example, a first enrichment reagent may be used to enrich a first population of cells positive for a first target moiety and a second enrichment reagent may be used to enrich a first population of cells positive for a second target moiety. Following, a first separation reagent may be used to enrich a second population of cells positive for the first target moiety, and a second separation reagent may be used to enrich a second population of cells positive for the second target moiety.

In the foregoing adaptation, the first enrichment reagent and second enrichment reagent will effect the specific enrichment of the first population of cells positive for the first target moiety and the first population of cells positive for the second target moiety, respectively. Accordingly, the first enrichment reagent and second enrichment reagent are different in at least one respect, such as in regard to the nature of the active ingredients respectively included therein. Likewise, the first separation reagent and second separation reagent will effect the specific enrichment of the second population of cells positive for the first target moiety and the second population of cells positive for the second target moiety, respectively. Accordingly, the first separation reagent and second separation reagent are different in at least one respect, such as in regard to the nature of the active ingredients respectively included therein.

The methods of this disclosure may further comprise culturing one, some or all of the segregated populations of cells in appropriate culture conditions. Such methods may comprise seeding the segregated population(s) of cells at an effective cell density and under effective culture conditions to expand such segregated population(s) of cells.

In one embodiment, segregated CD271^{High} cells may be cultured under conditions appropriate for neural crest cells, and segregated CD271^{Low} cells may be discarded or cultured under conditions appropriate for neuroectodermal cells.

If the cells are CD271^{High} cells the effective cell density may be greater than 1×10⁵ cells/cm². If the cells are CD271^{Low} cells the effective cell density may be greater than or less than 1×10⁵ cells/cm².

### Assays

In one aspect, assays of this disclosure encompass those steps for identifying, in a sample, distinct populations of cells positive for a target moiety.

As described above, the assays may begin by providing a sample of cells. The sample of cells may originate from any source, but in preferred embodiments, the sample is a single-cell suspension. In some embodiments, the sample of cells may include or consist of relatively small clusters or clumps of cells, or the sample of cells may include or consist of relatively large aggregates of cells.

In many cases, prior to providing the sample of cells, they may be pre-processed to break down tissues or organs into constituent parts, to clarify solutions comprising cells of certain contaminants therein, or to detach the cells from a substrate.

In embodiments where tissues or organs are the starting point, methods of dissociating the tissue or organ into single cells are known. For example, the tissue or organ may be digested enzymatically, disrupted mechanically, or disaggregated chemically. In some cases, processing a tissue or organ may involve any combination of enzymatic digestion, mechanical disruption, and chemical disaggregation to yield a suitable suspension of cells.

In embodiments where solutions comprising cells are the starting point, such solutions may be clarified to remove contaminants and the like. Methods of clarifying such solutions are known, and include filtration or chemical treatment. For example, if the starting cells are to be obtained from a blood sample it may need to be cleared of red blood cells and/or other types of cells of non-interest. In one embodiment, a blood sample may be clarified by density gradient centrifugation. In one embodiment, agglutinating red blood cells may clarify a blood sample, such as by performing a rosetting (RosetteSep™ STEMCELL Technologies) protocol. In one embodiment, a blood sample may be clarified by lysing red blood cells with a lysis buffer. In one embodiment, a blood sample may be clarified by changing the osmotic pressure within the red blood cells. If the solution comprising cells is a bodily fluid other than blood, such as urine, it may be important to concentrate the cells therein, while also clarifying the solution of non-cellular components.

In some embodiments, the starting cells may have previously been in culture. If grown in suspension (i.e. under non-adherent conditions), the cells may be ready for use in the methods disclosed herein. However, cells grown in suspension are typically bathed in a cell culture medium and it may be appropriate to pellet the cells and resuspend them in an appropriate physiological buffer, such as phosphate buffered saline or EasySep™ Buffer (STEMCELL Technologies). Further, a suspension of cells bathed in a cell culture medium may not be at an optimal density, which may be readily adjusted by centrifugation and resuspension, such as in an appropriate buffer.

In some embodiments, the starting cells may have been previously cultured adhered to a substrate, whether a tissue culture dish or flask, or to microcarriers. In such embodiments, it is necessary to detach the cells from the substrate, and those skilled in the art will know this may be done by mechanical, enzymatic, or chemical means. Whether the cells are mechanically, enzymatically, or chemically detached from a substrate, or detached using a combination of any of these methods, adjusting the density of the detached cells may also be necessary prior to initiating the methods disclosed herein. When detaching the cells, care should be taken to minimize or avoid excessive exposure to said agent because a target moiety (and/or other moieties) present on the surface of the cells may be digested.

The methods disclosed herein encompass a broad range of cell densities. In one embodiment, the cell density in the sample should be between about 1×10⁴ cells/mL and 1×10¹⁰ cells/mL, or between about 1×10⁵ cells/mL and 1×10⁹ cells/m L, or between about 5×10⁵ cells/mL and 5×10⁸ cells/mL. In one embodiment, the cell density in the sample should be about 5×10⁷±0.5×10⁷ cells/mL.

Once the sample of cells is suspended in an appropriate buffer and adjusted to an appropriate density, target moiety-positive cells within the sample are labeled with particles to form cell:particle complexes.

In one embodiment, the target moiety is a cell surface marker. Many cell surface markers are known, and are thus encompassed by this disclosure, provided that the target moiety may be bound by a binding member, such as an antibody or antibody fragment. Important for the assays disclosed herein, populations of cells within the sample are characterized by unique or distinguishable target moiety levels. For example, a target moiety level of a first population of cells (positive for a target moiety) is relatively lower than the target moiety level of a second population of cells (also positive for the target moiety).

In one embodiment, the target moiety is human CD271, wherein CD271^{High} distinguishes neural crest cells and CD271^{Low} distinguishes neuroectodermal and non-neural ectodermal cells.

In one embodiment, the target moiety is human CD49d, wherein CD49d^{High} distinguishes neural crest cells and CD49d^{Low} distinguishes neuroectodermal and non-neural ectodermal cells.

In one embodiment, the target moiety is human CD25, wherein CD25^{High} distinguishes regulatory T and activated T cells and CD25^{Low} distinguishes non-activated T cells.

In one embodiment, the target moiety is human CD8, wherein CD8^{High} distinguishes T cells and CD8^{Low} distinguishes a subset of natural killer cells.

In one embodiment, the target moiety is human CD56, wherein CD56^{High} distinguishes cytokine-producing subsets of natural killer cells and CD56^{Low} distinguishes cytotoxic subsets of natural killer cells.

In one embodiment, the target moiety is mouse CD138, wherein CD138^{High} distinguishes terminally differentiated plasmablasts and plasma cells and CD138^{Low} distinguishes pre-B cells.

It will be clear to the skilled reader that the methods disclosed herein are not limited to the target moieties specified above, but are rather generalizable to any system where different populations of cells may be distinguished based on a target moiety level.

Labeling the first population of cells positive for a target moiety and the second population of cells positive for the target moiety with particles (to form cell:particle complexes) may be accomplished in numerous ways. In one embodiment, the labeling, binding or connection between the particles and the first population of cells and the second population of cells, respectively, is intermediated by antibodies or antibody fragments. In one embodiment, the antibodies or antibody fragments comprise a particle-specific member and a target moiety-specific member.

Antibodies or antibody fragments of this disclosure may correspond to any structure capable of binding a target moiety. In one embodiment, the antibodies may correspond to any isotype, including IgA, IgD, IgE, IgG, and IgM. In one embodiment, the antibody fragments are F(ab), F(ab′)2, scFv fragments, or any adaptations thereof. In some embodiments, the antibodies or antibody fragments bind a target moiety with a high degree of specificity. Thus, it is preferred that the antibodies or antibody fragments are monoclonal.

In one embodiment, labeling of the target moiety with a particle is mediated by a single antibody, or fragment thereof. In such an embodiment, one arm of the single antibody or fragment thereof, binds the particle (i.e. the particle-specific member) and the other arm binds the target moiety (i.e. the target moiety-specific member).

In one embodiment, labeling of the target moiety with a particle is mediated by more than one antibody, or fragment thereof. In one embodiment, a particle-specific member is linked, directly or indirectly, to the target moiety-specific member. In one embodiment, a particle-specific member and the target-moiety-specific member form a bispecific complex.

In one embodiment, the bispecific complex includes a particle-specific member bound directly to a target moiety-specific member. In such an embodiment, a region of the particle-specific member may be bound directly to a region of the target moiety-specific member. For example, the Fc region of the particle-specific member may be directly conjugated to the Fc region of the target moiety-specific member. In the case of antibody fragments, a linking portion of the particle-specific member may be directly conjugated to a linking portion of the target moiety-specific member

In one embodiment, the bispecific complex includes a particle-specific member linked indirectly to a target moiety-specific member. In such an embodiment, indirect linkage of the particle-specific member and the target moiety-specific member can be accomplished in any way. For example, the particle-specific member and the target moiety-specific member may each be bound to a common element. For example, the common element may be a polymer capable of being conjugated with antibodies or antibody fragments. Or, the common element may be a particle or a bead. Or, the common element may be a complementary pair of entities, such as biotin and avidin/streptavidin or two oligonucleotides, where each entity is conjugated to one of the particle-specific member or the target moiety-specific member. In one embodiment, the particle-specific member and the target moiety-specific member may be linked in an immunological complex, wherein they are linked by one or more antibodies or antibody fragments. In one embodiment, the bispecific complex comprises two antibodies or F(ab′)2 fragments thereof—a particle-specific member and a target-moiety-specific member—that are linked in any way as described herein.

The assays may further comprise providing at least a saturating quantity of particles relative to the target moiety level (i.e. the sum of target moieties among the first population of cells positive for a target moiety and the second population of cells positive for the target moiety). Thus, a sufficient quantity of particles are available to label all target moiety-positive cells in a sample, which may enhance recoveries.

In one embodiment, the particles are coated with a polymer. In such an embodiment, the polymer may be PEG, PEG-based, or PEG-like. Or, the polymer may be Dextran, Dextran-based, or Dextran-like. Regardless of the nature of the polymer coating the particles, the particle-specific member should be specific for the polymer (of the particle) rather than the particle per se.

The particles may possess qualities that facilitate fractionating cell:particle complexes from other cells in a sample that are not positive for the target moiety. In one embodiment, the particles are responsive to a magnetic field. In such an embodiment, exposing cell:particle complexes (connected by antibodies or antibody fragments) formed in a sample to a magnetic field, as typically done in immunomagnetic separations, can fractionate the cell:particle complexes from other cells in the sample that are not positive for the target moiety. In one embodiment, the particles are responsive to a density of a solution in which the cells are suspended. For example, the particles may have a density that is lower than a density of the solution, in which case cell:particle complexes will float in the solution. Or, the particles may have a density that is higher than a density of the solution and cells not included in cell:particle complexes, in which case cell:particle complexes will sink in the solution. Thusly, cell:particle complexes can be fractionated from other cells in the sample that are not positive for the target moiety. The foregoing particles are available from various suppliers and/or manufacturers, including STEMCELL Technologies. Accordingly, an early or preliminary step of the assays disclosed herein may further comprise fractionating the cell:particle complexes from the sample after connecting particles and cells positive for a target moiety (via antibodies or antibody fragments).

Regardless of the other qualities of the particles, the particles sufficiently alter the readout (such as a side scatter readout) of the cells positive for the target moiety when labeled with a particle (such as by antibodies or antibody fragments, as described above). In one embodiment, a flow cytometry readout of cell:particle complexes is sufficiently sensitive to distinguish between cell:particle complexes comprising a first population of cells positive for a target moiety and cell:particle complexes comprising a second population of cells positive for the target moiety. In such an embodiment, a target moiety level of the first population of cells positive for the target moiety is relatively lower than the target moiety level of the second population of cells positive for the target moiety.

Accordingly, once the cell:particle complexes are formed, the assays comprise acquiring by flow cytometry a readout of the cell:particle complexes. Given that a target moiety level of the first population of cells positive for the target moiety is relatively lower than the target moiety level of a second population of cells positive for the target moiety, the readout of cell:particle complexes comprising the first population of cells is distinct from the readout of the second population of cells.

Furthermore, a readout of both the cell:particle complexes comprising a first population of cells positive for a target moiety and the cell:particle complexes comprising a second population of cells positive for the target moiety is distinct from the readout of uncomplexed target moiety-positive cells.

Further, in some embodiments, such as where the readout is a side scatter profile of the cell:particle complexes, the readout will show distinct populations of cells corresponding to their respective target moiety level. Thus, the assays may further comprise (after acquiring the read-out by flow cytometry) contacting the cell:particle complexes with an enrichment reagent (as described above) and reacquiring by flow cytometry the readout to assess a shift in the readout. A shift in the readout (such as a side scatter profile) may preferentially occur in relation to the first population of cells positive for the target moiety, indicating that such population is substantially delabeled. Similarly, the assays may further comprise contacting the cell:particle complexes with different concentrations of an enrichment reagent and assessing by a flow cytometry a shift in the readout corresponding to each concentration of the enrichment reagent. Accordingly, such an assay may be used to assess the dose-responsiveness of cell:particle complexes, particularly to specific populations of cells labeled with the particles, to an enrichment reagent (as previously described herein).

In one embodiment, the assays may further comprise gating the readout, such as a side scatter profile, on the target moiety.

In one embodiment, the assays may further comprise enriching the cells in the sample that are positive for the target moiety, and thus included within cell:particle complexes, before acquiring the readout, as described hereinabove. In one embodiment, enrichment of the cell:particle complexes in the sample may be performed by immunomagnetic cell separation, as described above.

### Kits

In another aspect of this disclosure are provided kits for carrying out the methods and assays disclosed hereon. Specifically, the kits of this disclosure may be used to enrich a first population of cells positive for a target moiety and/or a second population of cells positive for the target moiety from a sample.

In one embodiment, a kit includes a tube containing particles of this disclosure. In one embodiment, the particles included in the kits are polymer-coated. In one embodiment, the particles are coated with PEG or PEG derivative. In one embodiment, the particles are coated dextran or a dextran derivative.

In one embodiment, a kit also includes a tube containing antibody complexes of this disclosure. In one embodiment, the antibody complexes are bispecific complexes. In one embodiment, the bispecific complexes comprise a particle-specific member linked to a target moiety-specific member.

In one embodiment, a kit also includes a tube containing an enrichment reagent of this disclosure. In one embodiment, the enrichment reagent is PEG-containing or contains a PEG-derivative. In one embodiment, the enrichment reagent is dextran-containing or contains a dextran-derivative.

In one embodiment, a kit also includes a tube containing a separation reagent. In one embodiment, the separation reagent is PEG-containing or contains a PEG-derivative. In one embodiment, the separation reagent is dextran-containing or contains a dextran-derivative.

In one embodiment, a concentration of PEG (or PEG-derivative) or dextran (or dextran-derivative) is relatively lower in the enrichment reagent than in the separation reagent.

In one embodiment, a kit also includes instructions on how to perform the methods and assays as disclosed herein.

The following non-limiting examples are illustrative of the present disclosure.

### EXAMPLES

### Example 1: Differentiating CD271-Presenting Cells Using Different PSC Lines

Neural crest cells may spontaneously arise in neural and other differentiation protocols, but in other applications it may be desirable to differentiate starting cells into neural crest cells. When differentiating PSC to neural crest cells, the efficiency may be cell line-dependent.

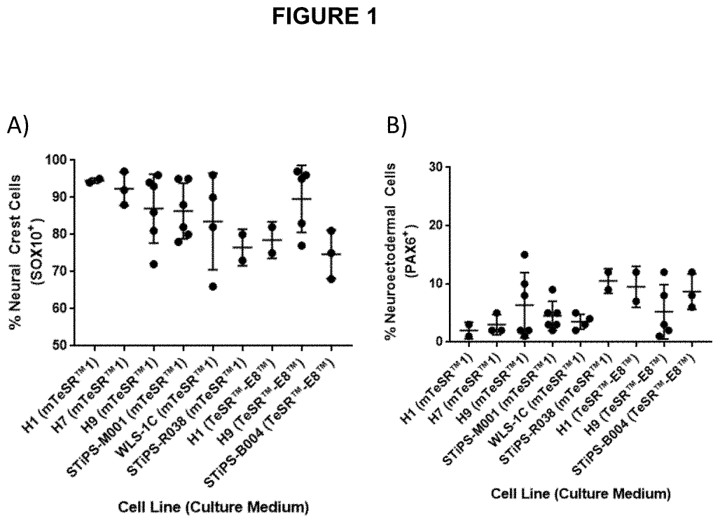

Various PSC lines were differentiated in the presence of STEMdiff™ Neural Crest Differentiation Kit (STEMCELL Technologies Part ID #08610) according to the manufacturer's protocol. FIG. 1 shows the efficiency of differentiating H1 ES cells, H7 ES cells, H9 ES cells, WLS-1C iPS cells, STiPS-M001 iPS cells, STiPS-6004 iPS cells, and STiPS-R038 iPS cells into CD271^{High} neural crest cells using the STEMdiff™ Neural Crest Differentiation Kit (STEMCELL Technologies).

After a differentiation protocol, the differentiated cells were washed with room temperature PBS (Ca²⁺- and Mg²⁺-free) or DMEM/F12, and incubated for 5-7 minutes at 37° C. in either 1 mL of pre-warmed (37° C.) 0.25% w/v Trypsin-EDTA or Accutase™, either of which may be purchased from STEMCELL Technologies. In the case where a Trypsin-containing solution is used it may need to be inactivated, such as by 1 mL of a pre-warmed (37° C.) 0.5% w/v Soybean Trypsin Inhibitor, ACF (STEMCELL Technologies). The cells may be dislodged using a serological pipette, and like samples may be pooled, prior to centrifugation at 300×g for 5 minutes (low brake setting) and resuspension in an appropriate buffer.

### Example 2: Flow Cytometry

Cells harvested in accordance with Example 1 were typically resuspended in an appropriate volume of a serum-free solution, such as RoboSep Buffer 2 (STEMCELL Technologies), or any other phosphate buffered saline-comprising solution. In this example, a composition of Dulbecco's phosphate buffered saline with 0.5% w/v bovine serum albumin and 2 mM EDTA was used to resuspend the cells to obtain a cell concentration of 2.5×10⁷ total cells/mL. Optionally, the harvested cells may be passed through a 70 um cell strainer to remove larger bodies prior to or after centrifuging the samples.

All flow cytometry was performed using up to 1×10⁶ cells stained with flourochrome-conjugated antibodies on a Beckman Coulter CytoFLEX instrument, and data analysis was performed using FCS Express, version 5. Cells were typically stained in a 96-well round-bottom plate.

Where intracellular staining was performed, typically with anti-SOX10 and/or anti-PAX6 fluorochrome-conjugated antibodies, cells were first fixed with 200 μL of cold 4% paraformaldehyde and incubated for about 15 minutes at 2-8° C. After fixation, the cells were centrifuged at 700×g for 3 minutes, resuspended in 250 μL of room temperature PBS+0.1% Tween20, and incubated for about 15 minutes at room temperature. After permeabilization, the cells were centrifuged at 700×g for 3 minutes, resuspended in 50 μl of RoboSep Buffer 2 (STEMCELL Technologies), and stained with 50 μL of a 2× staining cocktail of one or more antibodies. Samples were incubated in the dark for 15 minutes and then washed twice with RoboSep Buffer 2 (STEMCELL Technologies). After the final wash, the cells were resuspended with 100 μl of RoboSep Buffer 2 (STEMCELL Technologies).

### Example 3: Markers of Neural Crest Cells

Neural crest cells have been historically defined based on intracellular marker expression, such as SOX10, PAX7 and TFAP2. Due to the relatively large size of fluorochrome-conjugated antibodies used to detect these intracellular markers, cell fixation and permeabilization is required, which renders the cells non-viable. Neural crest cells may also be defined based on expression of surface antigens, such as CD57, CD271 and CD49d. Using surface-expressed antigens is advantageous for analyzing neural crest and other cells because they are not subjected to the harsh intracellular staining procedure and remain viable. Neural crest cells share surface-expression of some cell markers with developmentally related cell varieties of the ectoderm. One such example includes neuroectodermal cells, defined by the intracellular expression of the PAX6 marker, which are also identifiable by surface expression of the CD271 marker. However, the level of surface expression of these shared antigens can differ, such as with CD271. When assessed by flow cytometry, the PAX⁶⁺ neuroectodermal cells can have up to 10-fold fewer CD271 markers expressed on the cell surface when compared to SOX10⁺ neural crest cells.

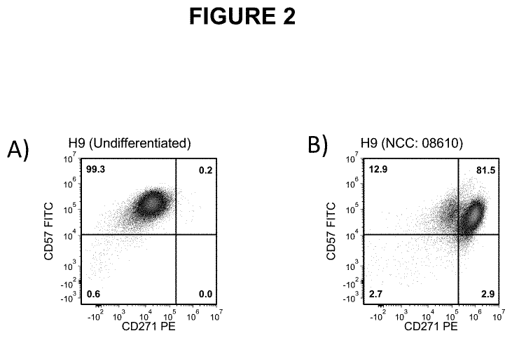

Undifferentiated PSC express relatively low levels of neural crest marker CD271, as assessed by flow cytometry (performed as described in Example 2) of H9 cells using PE-conjugated anti-CD271 antibody (FIG. 2A). After the cells are cultured as described in Example 1, increased CD271 stratifies two distinct populations of target moiety positive cells, CD271^{High} and CD271^{Low} (FIG. 2B).

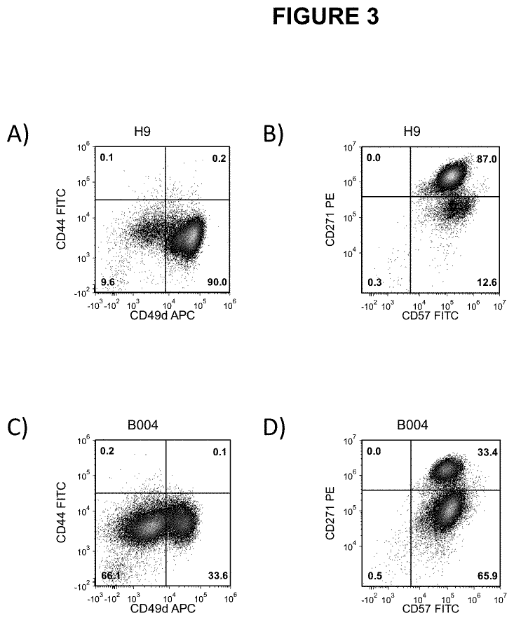

As with CD271, neural crest cells differentiated from H9 or B004 cells as described in Example 1 also stratify into two distinct populations of CD49d presenting cells, CD49d^{High} and CD49d^{Low} (FIG. 3).

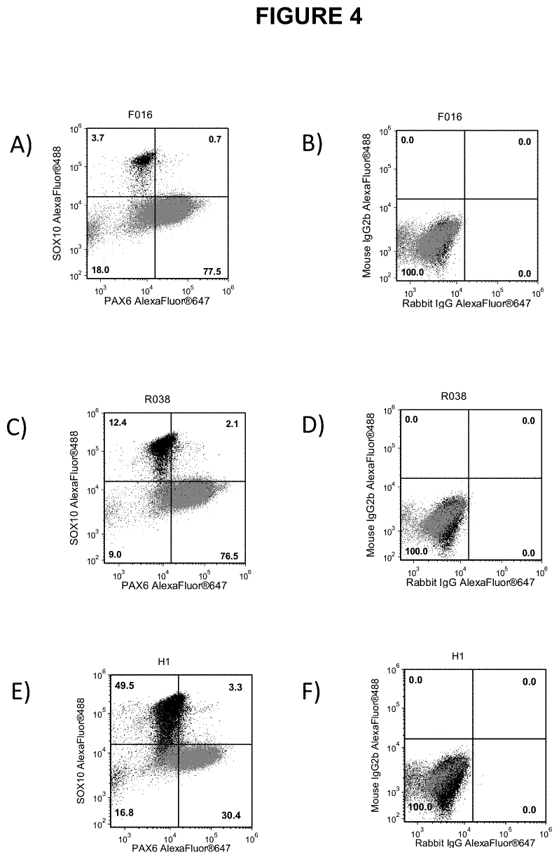

Intracellular staining (as described in Example 2) of differentiated PSC (as described in Example 1) was performed to correlate surface marker expression with intracellular markers of neural crest cells. SOX10 is a known marker of neural crest cells (Liu and Cheung (2016), Developmental Biology, 419, 199-216) while PAX6 is a known marker of neuroectodermal cells (Liu and Cheung (2016)). CD271⁺ cells differentiated from STiPS-F016 (iPS), R038 and H1 cells were stained with flourochrome-conjugated anti-SOX10 and anti-PAX6 antibodies, and analyzed by flow cytometry (as described in Example 2) (FIG. 4).

### Example 4: Preparation of Bispecific Complexes

Bispecific complexes were prepared by incubating target moiety-specific members, particle-specific members, and linker members in PBS. The concentrations of individual antibodies or antibody fragments may be varied; theoretically, about equimolar concentrations of the target moiety-specific members and the particle-specific members will yield the highest ratio of bispecific complexes. As described hereinabove, the disclosed subject matter is agnostic to the type of linker member, although in this case the linker member was an antibody having specificity for the target moiety-specific members and the particle-specific members. Following incubation, the formed bispecific complexes of antibodies (or antibody fragments) may be used in downstream assays, such as in cell separation protocols.

Where CD271⁺ cells are intended to be targeted by the bispecific complex, an anti-CD271 antibody and an antibody or antibody fragment against the polymer coating of the particle may be included in the initial incubation. Where CD56⁺ cells are intended to be targeted by the bispecific complex, an anti-CD56 antibody and an antibody or antibody fragment against the polymer coating of the particle may be included in the initial incubation. Where CD25⁺ cells are intended to be targeted by the bispecific complex, an anti-CD25 antibody and an antibody or antibody fragment against the polymer coating of the particle may be included in the initial incubation. Where CD127⁺ cells are intended to be targeted by the bispecific antibody complex, an anti-CD127 antibody and an antibody or antibody fragment against the polymer coating of the particle may be included in the initial incubation. Where CD8⁺ cells are intended to be targeted by the bispecific antibody complex, an anti-CD8 antibody and an antibody or antibody fragment against the polymer coating of the particle may be included in the initial incubation. Where mouse CD138⁺ cells are intended to be targeted by the bispecific antibody complex, an anti-CD138 antibody and an antibody or antibody fragment against the polymer coating of the particle may be included in the initial.

After the bispecific complexes have formed, they are incubated with a sample of cells, preferably a single-cell suspension, which may be obtained using conventional techniques. Following incubation of bispecific complexes and cells, particles are added to the sample. Herein, the particles are either EasySep Releasable RapidSpheres (STEMCELL Technologies, #50201) or EasySep Dextran RapidSpheres (STEMCELL Technologies, #50100). Thus, cells and particles are linked by the bispecific complexes, which cell:particle complexes may be separated based on the properties of the particles. Herein, the particles are at least responsive to a magnetic field, thereby enabling immunomagnetic separation of cells of interest (in a positive selection protocol).

### Example 5: Enrichment of Neural Crest Cells

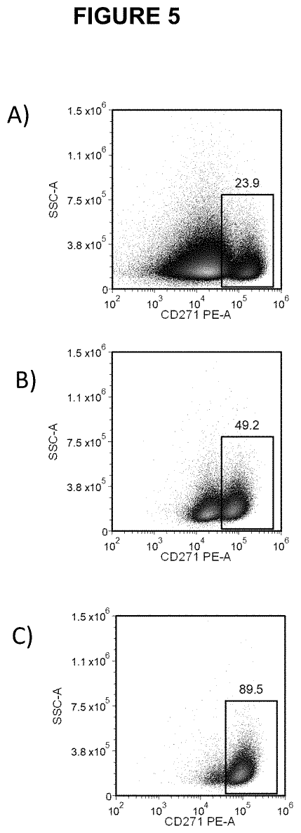

Neural crest cells were differentiated from 1C and H9 cells as described in Example 1. After 6 days of culture, the cells were harvested from culture plates as described in Example 2 and pooled into single-cell suspensions. Flow cytometry of the starting differentiated cell population was performed as described in Example 2 using a PE-conjugated anti-human CD271 antibody. The starting differentiated cell population included less than 25% CD271^{High} neural crest cells (FIG. 5A). After fractionating CD271⁺ cells from CD271⁻ cells by immunomagnetic separation, using an antibody complex as described in Example 4, the proportion of CD271^{High} neural crest cells increased to approximately 50% (FIG. 58). The proportion of CD271^{High} neural crest cells could be further increased to approximately 90% upon incubating the positively selected CD271⁺ cells with a 0.008% PEG enrichment reagent (FIG. 5C).

### Example 6: Components Used to Enrich Cells are Retained on the Cell Surface

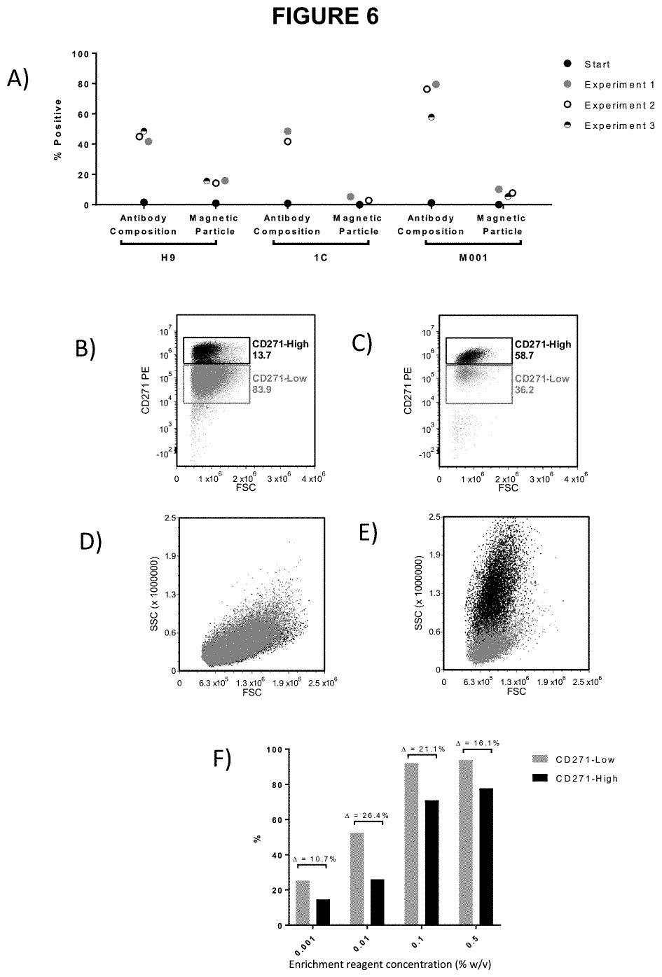

After fractionating the cells, such as CD271⁺ cells (as described in Example 5), they may subsequently be plated in an appropriate culture medium. In the case of CD271⁺ neural crest cells, they were replated in STEMdiff™ Neural Crest Differentiation Kit (STEMCELL Technologies) and cultured for an additional six days. Following, the cells were harvested as described in Example 1 and stained with a flourochrome-conjugated anti-isotype antibody (against the bispecific complex) and a fluorochrome-conjugated anti-particle antibody. The stained cells were analyzed by flow cytometry as described in Example 2, and both the antibody composition and the particles used to fractionate the CD271⁺ cells by immunomagnetic separation could be detected on a small proportion of cells (FIG. 6A). As previously discussed, neural crest cells differentiated from pluripotent stem cells (“PSC”) stratify into CD271^{High} and CD271^{Low} populations (FIG. 6B, for unlabeled cells; and FIG. 6C, for cells labeled with particles via antibody complexes). However, it was also shown that a side scatter profile of the cells labeled with particles may also resolve the different populations of CD271⁺ cells (FIG. 6E) in comparison to cells unlabeled by particles (FIG. 6D).

As enriched CD271⁺ cells may retain on their surface the particles used to carry out their fractionation, in certain applications it may be desirable to actively remove these components. After fractionating CD271⁺ cells in accordance with Example 5, the release efficiency of either CD271^{High} or CD271^{Low} from particles was tested using different concentrations of an enrichment reagent (STEMCELL Technologies, #17900) (FIG. 6F). Across all concentrations tested, the release efficiency of CD271^{Low} was higher than for CD271^{High}. However, at high concentrations of the enrichment reagent (e.g. 0.1%), almost all CD271⁺ cells are separated from the particles. Therefore, relatively lower concentrations of enrichment reagent, such as between about 0.001% and 0.1%, were taken as a starting point in optimization experiments.

### Example 7: Effects of Varying Enrichment Reagent Concentration on Delabeling CD271^{High} Cells

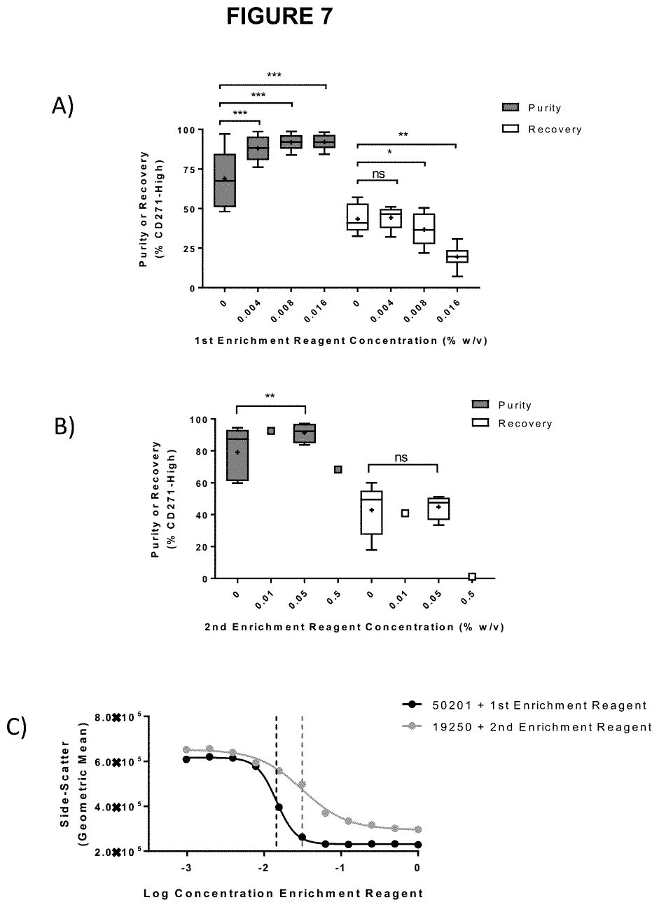

Further optimization of enrichment reagent concentration indicated that CD271^{High} cells could be further enriched from CD271⁺ cells upon addition of a low concentration of a PEG-containing enrichment reagent (“1^{st} Enrichment Reagent”). The average % purity of CD271^{High} cells in a sample of differentiated PSC was about 30% (not shown and FIG. 5A). The starting cell purity could be increased to an average of about 70% purity after isolating CD271⁺ cells, as described in Example 5 (FIG. 7A). Addition of either 0.004%, 0.008% or 0.016% first enrichment reagent during the enrichment protocol resulted in increased post-enrichment purity of CD271^{High} cells, indicating a reduction of CD271^{Low} contaminating cells. However, the use of relatively higher concentrations of enrichment reagent is detrimental to post-enrichment recovery of CD271^{High} cells (FIG. 7A).

Further optimization of enrichment reagent concentration indicated that CD271^{High} cells could be further enriched from CD271⁺ cells upon addition of a low concentration of dextran-containing enrichment reagent (“2^{nd} Enrichment Reagent”). The average % purity of CD271^{High} cells in a sample of differentiated PSC was about 30% (not shown and FIG. 5A). The starting cell purity could be increased to an average of about 80% purity after isolating CD271⁺ cells, as described in Example 5 (FIG. 7B). Addition of either 0.01%, 0.05% or 0.5% second enrichment reagent during the enrichment protocol resulted in increased post-enrichment purity of CD271^{High} cells, indicating a reduction of CD271^{Low} contaminating cells. However, the use of relatively higher concentrations of enrichment reagent is detrimental to post-enrichment recovery of CD271^{High} cells (FIG. 7B).

A rapid and accurate flow cytometry assay was developed to screen for optimal concentrations of enrichment reagent. Briefly, a population of cells is first incubated, as described in Example 4, with an bispecific complexes having specificity for both target moieties of the cells of interest and a particle to allow binding of target moiety-positive cells. Following, such complexes were incubated with particles having high side scatter, to form cell:particle complexes. High-side scatter particles can be defined as producing a geometric mean fluorescent intensity of approximately 2.5×10⁴ on a Beckman Coulter CytoFLEX instrument when acquired without cells and antibodies present. Thus, the side scatter of cell:particle complexes can produce a geometric mean fluorescent intensity signal of 1×10⁵ or higher, depending on the original scatter profile of the unlabeled population. Excess bispecific complexes and particles are washed, and then cells are stained with fluorochrome-conjugated antibodies. After a second wash to remove excess fluorochrome-conjugated antibodies, the cell:particle complexes are incubated with various concentrations of enrichment reagent. Due to the high side scatter of cell:particle complexes it is possible to assess by flow cytometry the release efficiency of a given concentration of enrichment reagent by a shift in side scatter. The geometric mean of the side scatter signal for cells labeled with particles was determined by flow cytometry analysis (FIG. 7C). Dose-response curves were generated using two formulations of enrichment reagent: a first PEG-containing enrichment reagent; and a second dextran-containing enrichment reagent. The dose-response curve was generated by plotting log[inhibitor] vs response using a variable slope fit (four parameter).

Reduction of side scatter is correlated with delabeling a population of cells (i.e. removing particles therefrom). An average of 2.5-fold decrease in SSC signal from the maximum side scatter signal represents a sufficient delabeling and reduced ability of the cells to migrate toward a magnetic field during immunomagnetic separation, allowing such cells to be poured out in the negative fraction.

### Example 8: Enrichment Reagent May be Added at any Time of Cell Enrichment

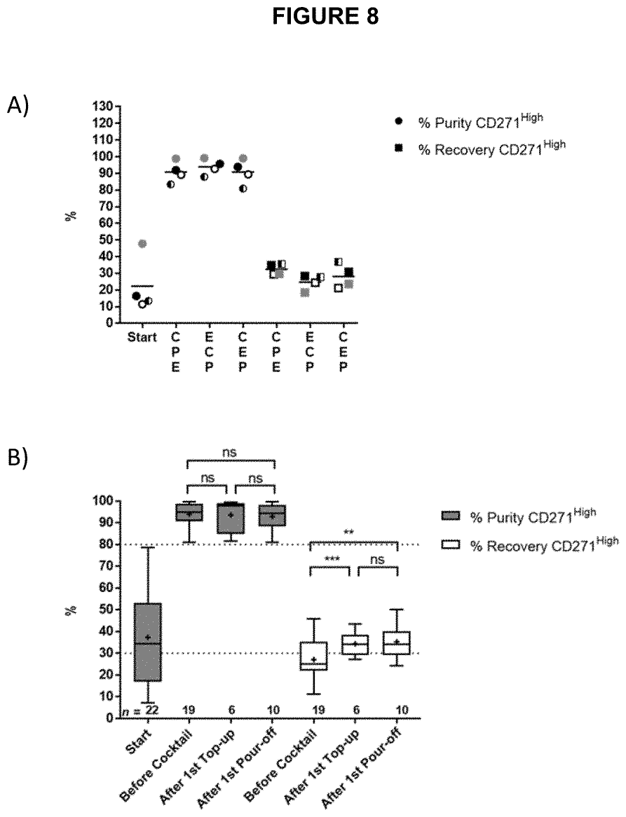

The timing when the enrichment reagent may be added during an enrichment protocol was investigated. In a cell separation protocol as described in Example 4, the sequence in which the antibody composition (“C”), the particles (“P”), and the enrichment reagent (“E”) are added to the sample of cells was varied. The order of addition does not appear to affect either average % purity or average % recovery of CD271^{High} cells (FIG. 8A).

In addition, the average % purity of enriched CD271^{High} cells did not appear to change whether the enrichment reagent was added before the addition of antibody composition, after the sample is incubated with antibody composition and particles (“after 1^{st} top-up”), or after the first magnetic separation (“after 1^{st} pour-off”) (FIG. 8B). However, the average % recovery of CD271^{High} cells appeared to increase when the enrichment reagent was added either after 1^{st} top-up or after 1^{st} pour-off (FIG. 8B).

### Example 9: Enrichment Reagent May be Used to Segregate CD271^{Low} Cells from CD271^{High} Neural Crest Cells

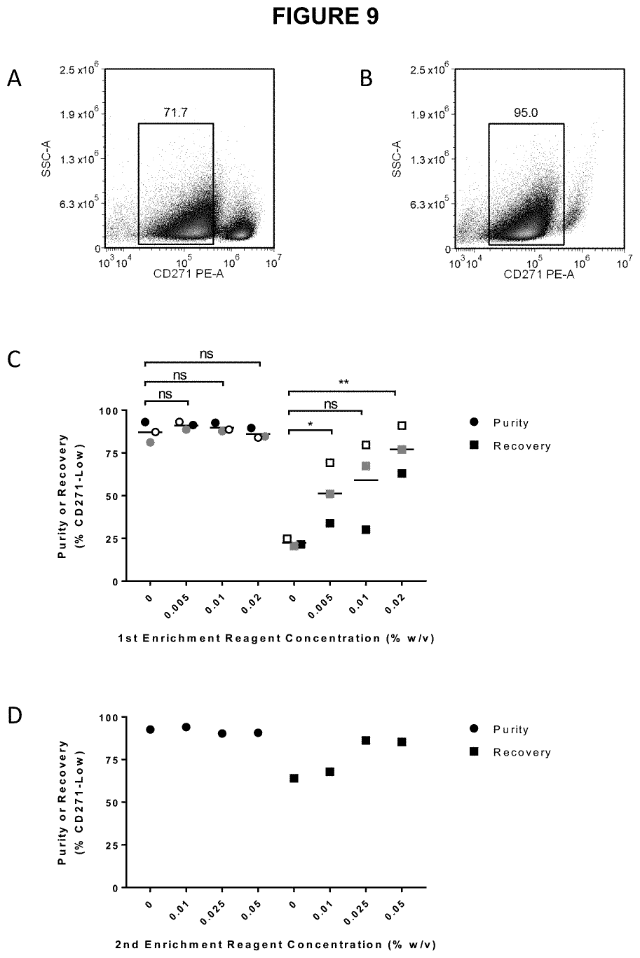

Neural crest cells were differentiated from 1C and H9 cells as described in Example 1. After 6 days of culture, the cells were harvested from culture plates as described in Example 2 and pooled into single-cell suspensions. Flow cytometry of the starting differentiated cell population was performed as described in Example 2 using a PE-conjugated anti-human CD271 antibody for detection. The starting differentiated cell population included approximately 70% CD271^{Low} cells (FIG. 9A). After a first fractionation of CD271⁺ cells as described in Example 5—and using anti-CD271 bispecific complexes as generated in Example 4—followed by incubation in a PEG-containing enrichment reagent, the proportion of CD271^{Low} cells in the negative pour-off fraction increased to approximately 95% (FIG. 9B). A slight increase in average purity and a marked increase in average recovery of CD271^{Low} cells is observed as the concentration of first enrichment reagent is increased (FIG. 9C). A substantially identical procedure as described in (B) and (C) was performed, but utilized EasySep Dextran RapidSpheres (STEMCELL Technologies) in immunomagnetic separations. After fractionating the differentiated CD271⁺ cells, they were incubated with different concentrations of a dextran-containing enrichment reagent, and the purity and recovery of CD271^{Low} cells were compared to those obtained with a control wash reagent, the wash reagent typically used in EasySep™ protocols (“0%”) (D).

### Example 10: Segregated CD271^{High} Neural Crest Cells are Functional

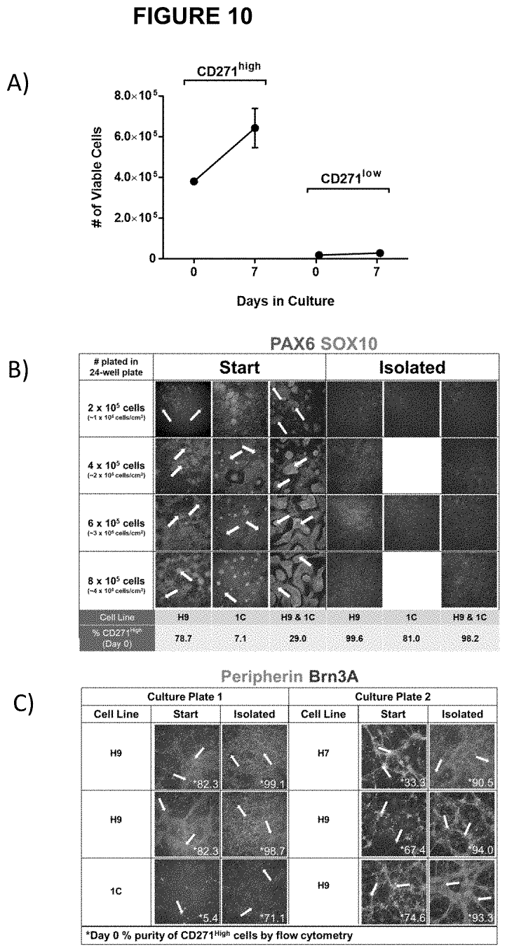

CD271^{High} neural crest cells enriched from a cell population and segregated from CD271^{Low} cells in enrichment reagent—and using anti-CD271 bispecific complexes as generated in Example 4—may be further cultured and/or maintained in STEMdiff™ Neural Crest Differentiation Kit (STEMCELL Technologies). The enriched CD271^{High} cells were replated (Day 0) and left in culture for 7 days. On Day 7 the culture of cells was harvested, counted, and analyzed by flow cytometry as described in Example 2 to quantify the number of CD271^{High} and CD271^{Low} cells. FIG. 10A shows that CD271^{High} cells exhibit robust expansion during the 7-day culture period, whereas CD271^{Low} cells exhibit low or no expansion.

Further, CD271^{High} neural crest cells enriched from a cell population and segregated from CD271^{Low} cells in enrichment reagent— and using anti-CD271 bispecific complexes as generated in Example 4— may be further cultured at cell densities optimal for establishing neural crest cells. In this example, optimal seeding density for neural crest cell populations is observed at a viable cell density of 2×10⁵ cells/cm² up to a maximum at 4×10⁵ cells/cm² (FIG. 10B). In the same example, a reduction of contaminating PAX6⁺ cell populations, indicated by white arrows, can be observed as the purity of the seeded CD271^{High} cells is increased via the disclosed enrichment procedure (FIG. 10B).

Further, CD271^{High} neural crest cells enriched from a cell population and segregated from CD271^{Low} cells in enrichment reagent—and using anti-CD271 bispecific complexes as generated in Example 4— may be further cultured in conditions to promote differentiation into peripheral neurons. In this example, the differentiation protocol of Lee G et al. (2010) Nat Protoc 5(4): 688-701 was utilized. CD271^{High} neural crest cells cultured in conditions that promote differentiation into peripheral neurons expressing peripherin and Brn3A (FIG. 10C).

### Example 11: CD25^{High} Cells May be Preferentially Enriched from Leukapheresis Samples

Leukapheresis samples (STEMCELL Technologies Part ID #70500) were processed first by lysing red blood cells using Ammonium Chloride Solution (STEMCELL Technologies Part ID #07850) according to the product information sheet. Samples were washed twice using EasySep™ Buffer (STEMCELL Part ID #20144) by topping samples up to 50 mL and centrifugation at 150×g for 10 minutes (no brake setting). Supernatant was removed by pipette and the cell pellet was resuspended in EasySep™ buffer prior to a CD25^{High} enrichment protocol essentially as described in Example 5.

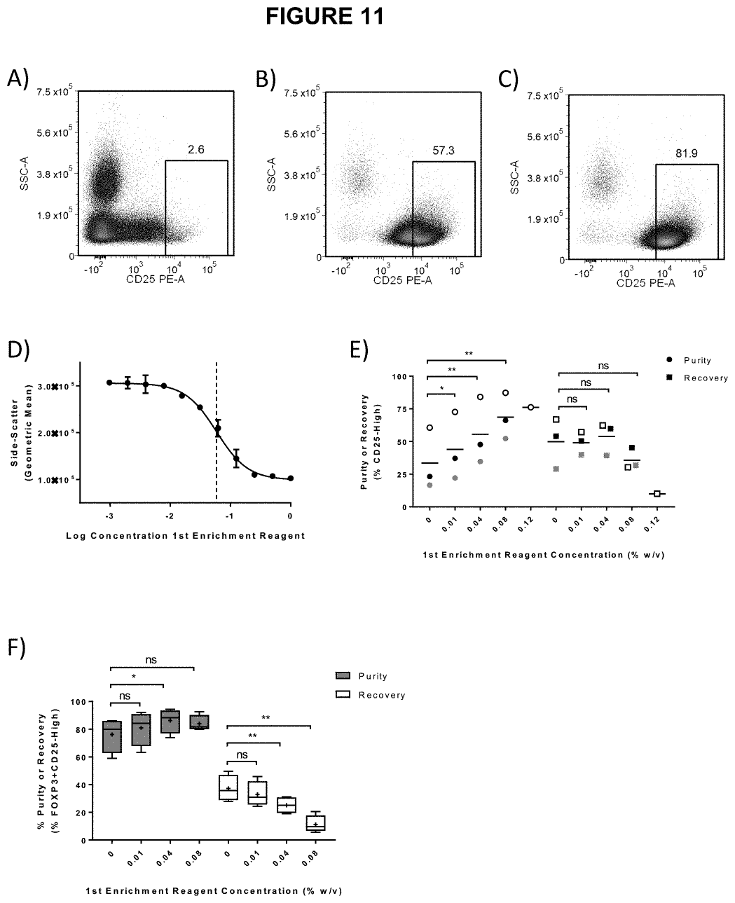

Flow cytometry of the processed leukapharesis sample was performed as described in Example 2 using a PE-conjugated anti-human CD25 antibody for detection. The starting cell population included about 2% CD25^{High} cells (FIG. 11A). After a first fractionation of CD25⁺ cells using anti-CD25 bispecific complexes as described in Example 4, the proportion of CD25^{High} cells increased to approximately 57% (FIG. 11B). The proportion of CD25^{High} cells could be further increased to approximately 82% upon incubating the fractionated cells with a PEG-containing enrichment reagent (FIG. 11C).

An appropriate concentration of enrichment reagent, as indicated by the dashed line (IC₅₀), could be determined by the assay described in Example 7 (FIG. 11D).

With increased concentration of enrichment reagent, the average CD25^{High} cell purity increased and only higher concentrations of enrichment reagent were detrimental to average CD25^{High} cell recovery (FIG. 11E).

After segregating CD25^{High} cells from CD25^{Low} cells using the enrichment reagent, the CD25^{High} cells were incubated with anti-CD127 bispecific complexes, formed essentially as described in Example 4. This CD127 depletion procedure is similar to that described in the EasySep™ Human CD4+CD127^{Low}CD25⁺ Regulatory T Cell Isolation Kit (STEMCELL Technologies Part ID #18063). After enriching for CD25^{High} cells using first enrichment reagent as described in Example 7, removal of CD127⁺ cells from the CD25^{High} population led to increased average FOXP3+CD25^{High} regulatory T cell purity (FIG. 11F). Progressively higher concentrations of first enrichment reagent used to enrich the CD25^{High} population had a progressively more detrimental effect on average FOXP3+CD25^{High} regulatory T cell recovery after CD127⁺ cell removal (FIG. 11F).

### Example 12: CD56^{High} Cells May be Preferentially Enriched from Leukapharesis Samples

Leukapheresis samples (STEMCELL Technologies Part ID #70500) were processed first by lysing red blood cells using Ammonium Chloride Solution (STEMCELL Part ID #07850) according to the product information sheet. Samples were washed twice using EasySep™ Buffer (STEMCELL Part ID #20144) by topping samples up to 50 mL and centrifugation at 150×g for 10 minutes (no brake setting). Supernatant was removed by pipette, with care taken not to disturb the cell pellet. Samples were suspended in EasySep™ buffer prior to performing a CD56^{High} enrichment protocol essentially as described in Example 5.

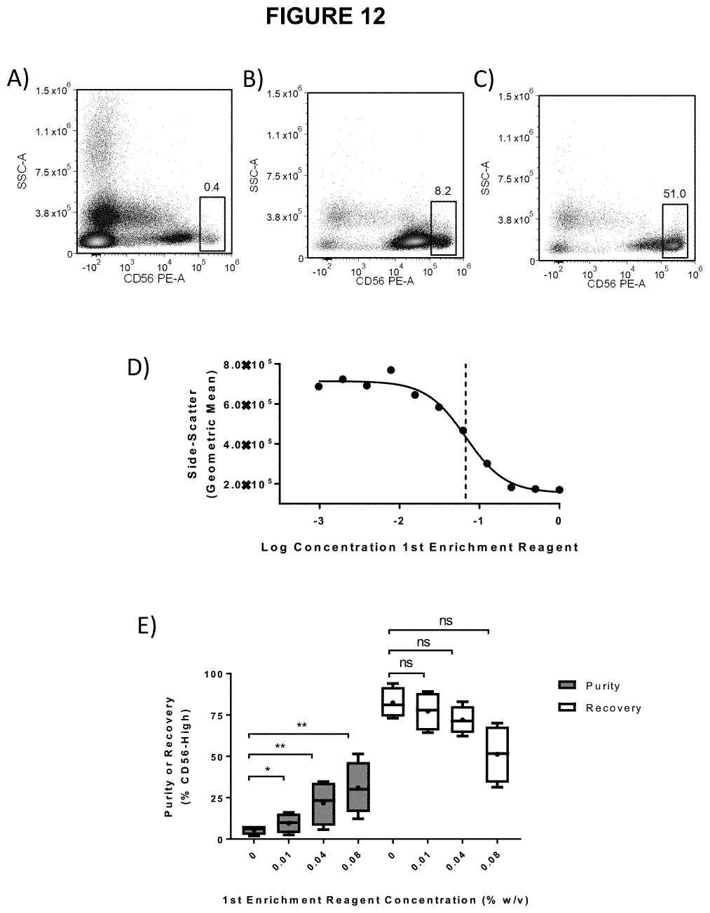

Flow cytometry of the processed leukapharesis sample was performed as described in Example 2 using a PE-conjugated anti-human CD56 antibody for detection. The starting cell population included about 0.5% CD56^{High} cells (FIG. 12A). After fractionating CD56⁺ cells using anti-CD56 bispecific complexes (as described in Example 4) in an immunomagnetic separation, the proportion of CD56^{High} cells increased to approximately 8% (FIG. 12B). The proportion of CD56^{High} cells could be further increased to approximately 50% upon incubating the fractionated sample in a PEG-containing enrichment reagent (FIG. 12C).

An appropriate concentration of first enrichment reagent, as indicated by the dashed line (IC₅₀), could be determined by the assay described in Example 7 (FIG. 12D).

With increased concentration of enrichment reagent, the average CD56^{High} cell purity increased, but progressively higher concentrations of the enrichment reagent had a progressively more detrimental effect on average CD56^{High} cell recovery (FIG. 12E).

### Example 13: CD8^{High} Cells May be Preferentially Enriched from Leukapharesis Samples

Leukapheresis samples (STEMCELL Technologies Part ID #70500) were processed first by lysing red blood cells using Ammonium Chloride Solution (STEMCELL Part ID #07850) according to the product information sheet. Samples were washed twice using EasySep™ Buffer (STEMCELL Part ID #20144) by topping samples up to 50 mL and centrifugation at 150×g for 10 minutes (no brake setting). Supernatant was removed by pipette and the cell pellet was resuspended in EasySep™ buffer prior to a CD8^{High} enrichment protocol essentially as described in Example 5.

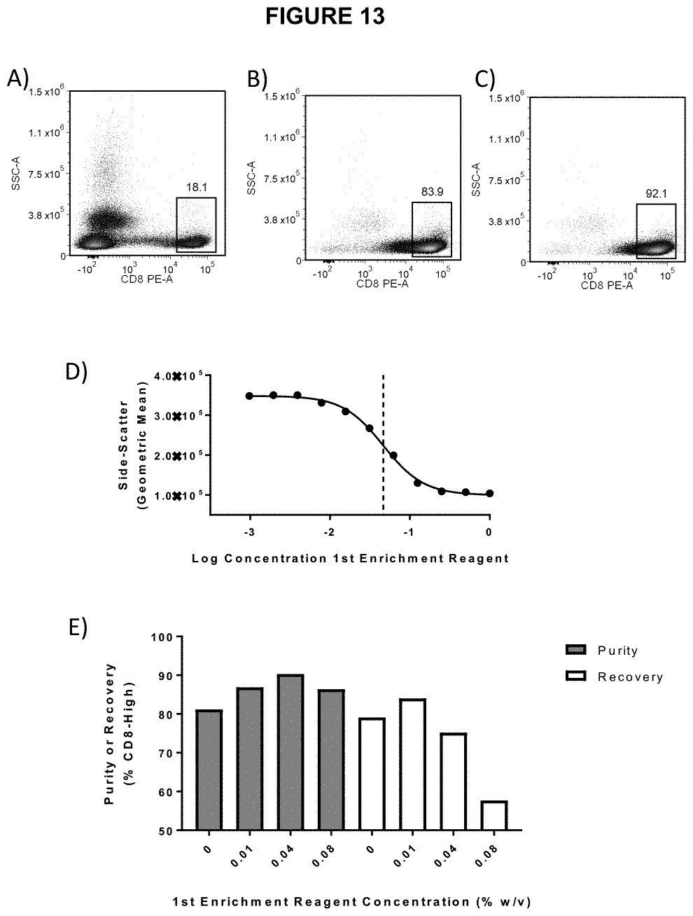

Flow cytometry of the processed leukapharesis sample was performed as described in Example 2 using a PE-conjugated anti-human CD8 antibody for detection. The starting cell population included about 8% CD8^{High} cells (FIG. 13A). After fractionating CD8⁺ cells using anti-CD8 bispecific complexes (as described in Example 4) in immunomagnetic separation, the proportion of CD8^{High} cells increased to approximately 84% (FIG. 13B). The proportion of CD8^{High} cells could be further increased to approximately 92% upon incubating the fractionated cells with a PEG-containing enrichment reagent (FIG. 13C).

An appropriate concentration of first enrichment reagent could be determined, as indicated by the dashed line (IC₅₀), by the assay described in Example 7 (FIG. 13D).

With increased concentration of enrichment reagent average CD8^{High} cell purity generally increased, but progressively higher concentrations of the enrichment reagent generally had a progressively more detrimental effect on average CD8^{High} cell recovery (FIG. 13E).

### Example 14: CD138^{High} Cells May be Preferentially Enriched from Naïve Mouse Splenocyte Samples

The spleen from a naïve C57BL/6 mouse was removed and mechanically dissociated through a 70 μm nylon mesh cell strainer into a 50 mL tube. Using EasySep™ Buffer (STEMCELL Part ID #20144) kept at 2-8° C., the volume of disaggregated spleen suspension was adjusted to 50 mL prior to centrifugation at 300×g for 10 minutes (low brake setting). The supernatant was removed and the cell pellet was suspended in EasySep™ Buffer. The single-cell suspension was kept on ice prior to a CD138^{High} enrichment protocol essentially as described in Example 5.

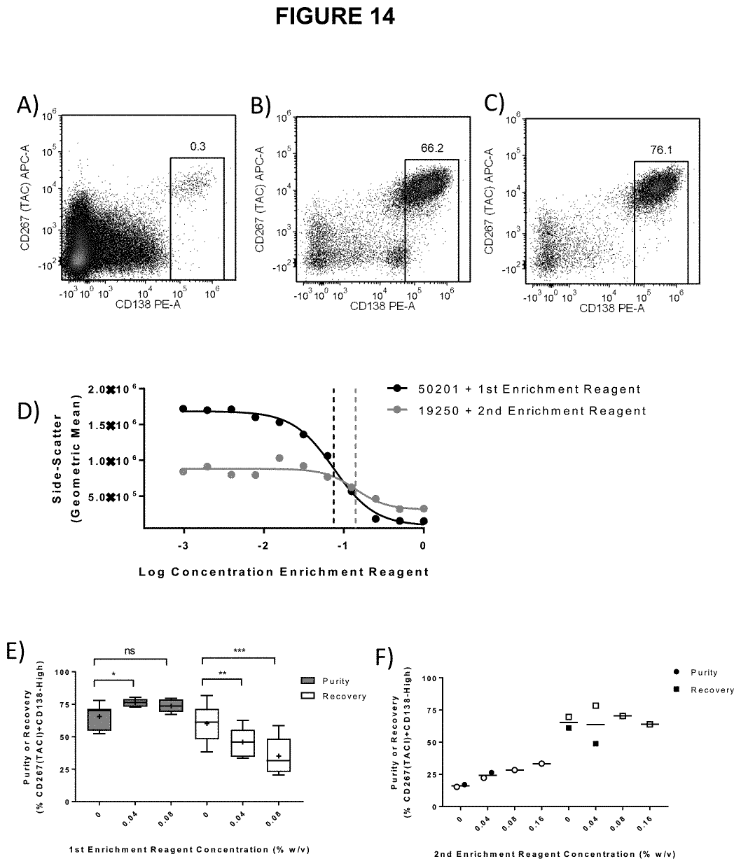

Flow cytometry of the isolated splenocyte cell populations was performed as described in Example 2 using an APC-conjugated anti-mouse CD267 (TACI) antibody and a PE-conjugated anti-mouse CD138 antibody for detection. The starting cell population included less than 1% CD138^{High} cells (FIG. 14A). After fractionating CD138⁺ cells using anti-CD138 bispecific complexes (as described in Example 4) in immunomagnetic separation, the proportion of CD138^{High} cells increased to approximately 65% (FIG. 14B). The proportion of CD138^{High} cells could be further increased to approximately 76% upon incubating the fractionated cells in a PEG-containing enrichment reagent (FIG. 14C).

An appropriate concentration of a first PEG-containing enrichment reagent and a second dextran-containing enrichment reagent could be determined, as indicated by the dashed line (IC₅₀), by the assay described in Example 7 (FIG. 14D).

First enrichment reagent increased average CD138^{High} cell purity, but higher concentrations of the enrichment reagent were detrimental to average CD138^{High} cell recovery (FIG. 14E).

Second enrichment reagent increased average CD138^{High} cell purity, and progressively higher concentrations of the enrichment reagent did not appear to have an effect on average CD138^{High} cell recovery (FIG. 14F).

### Example 15: CD138^{High} Cells May be Preferentially Enriched from Naïve Mouse Bone Marrow Samples

The femurs and tibias from a naïve C57BL/6 mouse were removed, mechanically dissociated using a mortar and pestle, and then passed through a 70 μm nylon mesh cell strainer into a 50 mL tube. Using EasySep™ Buffer (STEMCELL Part ID #20144) kept at 2-8° C., the volume of bone marrow cell suspension was adjusted to 50 mL prior to centrifugation at 300×g for 10 minutes (low brake setting). The supernatant was removed and the cell pellet was suspended in EasySep™ Buffer. The single-cell suspension was kept on ice prior to a CD138^{High} enrichment protocol essentially as described in Example 5.

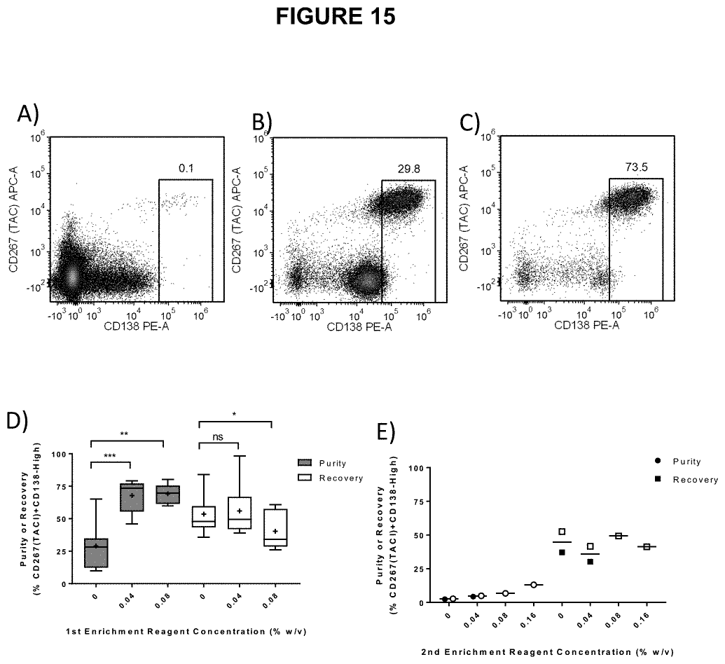

Flow cytometry of the isolated bone marrow cell populations was performed as described in Example 2 using an APC-conjugated anti-mouse CD267 (TACI) antibody and a PE-conjugated anti-mouse CD138 antibody for detection. The starting cell population included less than 1% CD138^{High} cells (FIG. 15A). After fractionating CD138⁺ cells using anti-CD138 bispecific complexes (as described in Example 4) in immunomagnetic separation, the proportion of CD138^{High} cells increased to approximately 30% (FIG. 15B). The proportion of CD138^{High} cells could be further increased to approximately 75% upon incubating the fractionated cells with a PEG-containing enrichment reagent (FIG. 15C).

A PEG-containing first enrichment reagent increased average CD138^{High} cell purity, but only the highest concentrations of enrichment reagent were detrimental to average CD138^{High} cell recovery (FIG. 15D).

A dextran-containing second enrichment reagent increased average CD138^{High} cell purity, and progressively higher concentrations of the enrichment reagent did not appear to have an effect on average CD138^{High} cell recovery (FIG. 15E).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of enriching a first population of cells positive for a target moiety and/or a second population of cells positive for the target moiety from a sample, the method comprising:
a) labeling the first population and the second population with particles to form cell:particle complexes;
b) contacting the cell:particle complexes with an enrichment reagent to substantially delabel the first population from the particles; and
c) isolating the first population from the sample,
wherein a level of the target moiety among the first population of cells is lower than the level of the target moiety level among the second population of cells.

2. The method of claim 1, further comprising:
d) contacting residual cell:particle complexes in the sample with a separation reagent to substantially separate the second population from the particles.

3. The method of claim 2, further comprising:
e) isolating the second population from the sample.

4. The method of claim, wherein the target moiety is a cell surface marker.

5. The method of claim 4, wherein the cell surface marker is human CD271, human CD25, human CD49d, mouse CD138, human CD8, or human CD56.

6. The method of claim 1, wherein the particles are coated with a polymer.

7. The method of claim 1, wherein the particles are responsive to a magnetic field.

8. The method of claim 1, further comprising fractionating the cell:particle complexes from the sample after step a) and before step c).

9. The method of claim 1, further comprising providing at least a saturating quantity of particles relative to the level of the target moiety of the first population and the second population.

10. The method of claim 1, wherein the labeling of the first population of cells or the second population of cells to the particles is intermediated by antibodies or antibody fragments.

11. The method of claim 10, wherein the antibodies or antibody fragments comprise a particle-specific member and a target moiety-specific member.

12. The method of claim 11, wherein the particle-specific member is linked, directly or indirectly, to the target moiety-specific member.

13. The method of claim 11, wherein the particle-specific member and the target-moiety-specific member form a bispecific complex.

14. The method of claim 2, wherein the enrichment reagent is formulated differently from the separation reagent.

15. The method of claim 14, wherein the enrichment reagent and the separation reagent each include a polymer.

16. The method of claim 15, wherein a concentration of the polymer is relatively lower in the enrichment reagent compared to the separation reagent.

17. The method of claim 15 or 16, wherein the polymer is PEG (polyethylene glycol), PEG-based, or PEG-like, or the polymer is dextran, dextran-based, or dextran-like.

18-34. (canceled)

35. A kit for enriching a first population of cells positive for a target moiety and/or a second population of cells positive for the target moiety from a sample, the kit comprising:
a) a tube containing polymer-coated particles;
b) a tube containing an antibody composition, the antibody composition comprising a particle-specific member linked to a target moiety-specific member;
c) a tube containing an enrichment reagent; and
d) optionally, a tube containing a separation reagent.

36. The kit of claim 35, wherein the enrichment reagent is PEG-containing or dextran-containing.

37. The kit of claim 35, wherein the separation reagent is PEG-containing or dextran-containing.

38. (canceled)
